(19) United States
(12) Patent Application Publication
(10) Pub. No.: US 2018/0311356 A1
(43) Pub. Date: Nov. 1, 2018

(54) METHOD TO INHIBIT ENDOTHELIAL-TO-MESENCHYMAL TRANSITION

(71) Applicants: THE CHILDREN'S MEDICAL CENTER CORPORATION, Boston, MA (US); THE GENERAL HOSPITAL CORPORATION, Boston, MA (US); THE BRIGHAM AND WOMEN'S HOSPITAL, INC., Boston, MA (US)

(72) Inventors: Joyce E. BISCHOFF, Weston, MA (US); Robert A. LEVINE, Brookline, MA (US); Elena AIKAWA, Chestnut Hill, MA (US)

(73) Assignees: THE CHILDREN'S MEDICAL CENTER CORPORATION, Boston, MA (US); THE GENERAL HOSPITAL CORPORATION, Boston, MA (US); THE BRIGHAM AND WOMEN'S HOSPITAL, INC., Boston, MA (US)

(21) Appl. No.: 15/769,443

(22) PCT Filed: Oct. 19, 2016
(86) PCT No.: PCT/US16/57676

§ 371 Date: Apr. 19, 2018

Related U.S. Application Data
(60) Provisional application No. 62/243,473, filed on Oct. 19, 2015.

(51) Int. Cl. A61K 45/00; A61P 9/00

(52) U.S. Cl. CPC A61K 45/00; A61P 9/00

(57) ABSTRACT

Embodiments herein provide for compositions and methods for inhibiting pathological endothelial-to-mesenchymal transition (EndMT), e.g., in endothelial cells and in a mammal. Specially, pathological EndMT can be inhibited by inhibiting CD45, either protein activity or protein expression or both, and optionally also inhibiting TGF-β. Compositions comprising a CD45 inhibitor, a CD45 inhibitor and a TGF-β inhibitor, or a CD45 inhibitor, a TGF-β inhibitor and an anti-fibrosis agent are disclosed.

### CROSS REFERENCE TO RELATED APPLICATION

This application claims benefit under 35 U.S.C. § 119(e) of the U.S. provisional application No. 62/243,473 filed Oct. 19, 2015, the content of which is incorporated herein by reference in its entirety.

### GOVERNMENT SUPPORT

This invention was made with Government support under Grant No.: RO1HL109506 awarded by the National Institutes of Health. The Government has certain rights in the invention.

### SEQUENCE LISTING

The instant application contains a Sequence Listing which has been submitted electronically in ASCII format and is hereby incorporated by reference in its entirety. The ASCII copy, created on Oct. 17, 2016, is named 701039-085931-PCT_SL.txt and is 1,025 bytes in size.

### FIELD OF THE DISCLOSURE

This disclosure relates to compositions and methods for blocking the endothelial-to-mesenchymal transition (EndMT) in endothelial cells where such transition produces pathological remodeling and fibrosis that lead to organ and tissue failure.

### BACKGROUND

Endothelial to mesenchymal transition (EndMT) is a normal process that occurs during development and it also occurs at a low level throughout life. During this process, the endothelial cells (ECs) lose their polarity and cell-to-cell contacts, and undergo a dramatic remodeling of the cytoskeleton. During this transition process from ECs to mesenchymal cells, there is a marked decrease in the expression of endothelial markers and concurrent increase in the expression of mesenchymal markers including smooth muscle α-actin (SMA), fibroblast-specific protein 1 (FSP1; also known as S100A4), fibronectin, and collagens. Furthermore, the newly transformed mesenchymal cells manifest migratory and proliferative phenotypes. Thus, the ECs become mesenchymal cells.

There are now an increasing number of instances where EndMT (also called EMT or EndoMT in some scientific papers) occurs in disease settings, and appears to contribute to the vascular, tissue and organ pathologies. For example, many of heart injuries end up in a common final pathway of pathologic tissue remodeling and fibrosis (defined by deposition of collagens, elastin, tenascin, and other matrix proteins), leading to the development of heart failure. Myocardial fibrosis induced by cardiac fibroblasts plays a dual role in cardiac remodeling after injury. While fibrosis plays important roles in wound healing, it also contributes to ventricular stiffening and heart failure progression. Recent reports have revealed that cardiac fibroblasts originate through the EndMT.

Several lines of evidence suggest that cardiac fibroblasts are a heterogeneous population and derive from various distinct tissue niches in physiological and pathological conditions. During embryonic heart development, cardiac fibroblasts are differentiated from epicardium or endocardium of the heart. In a healthy adult heart, cardiac fibroblasts reside in the interstitial tissue within the myocardium. Some reports have shown that heart-resident cardiac fibroblasts are the major source of tissue fibrosis associated with ischemic heart failure and hypertrophy. In addition, fibroblasts originated from bone marrow-derived cells including CD45-positive hematopoietic stem cells (HSCs) have also been shown to significantly contribute to remodeling of the injured heart. Finally, emerging evidence suggests that a subset of cardiac fibroblasts is originated from ECs in a mouse model of pressure overload. This endothelial mesenchymal transition has common features with epithelial mesenchymal transition. Taken together, cardiac fibroblasts are thought to be derived from resident fibroblasts, bone marrow-derived cells, and ECs.

Ischemic mitral regurgitation, (IMR) a common complication after myocardial infarction (MI) that doubles mortality. IMR induces adaptive cellular responses in the mitral valve (MV) that may be initially beneficial, but eventually lead to leaflet fibrosis and MV dysfunction. IMR is caused by left ventricular remodeling and dysfunction, which lead to papillary muscle displacement and tethering of the mitral valve (MV), restricting leaflet closure. Tethered MVs adapt by increasing their surface area, but this adaptation is often insufficient and appears to result in stiff, fibrotic valves, which may ultimately contribute to increased IMR. Several cellular events occur in the IMR MV: MV endothelial cells (mitral VECs) undergo endothelial-to-mesenchymal transition (EndMT), MV interstitial cell activate into myofibroblasts, and there is evidence for valve neovascularization and leukocyte infiltration. Infiltrating macrophages and leukocytes are known to release growth factors and cytokines, such as TGFβ family members, which promote angiogenesis, collagen production and attract additional inflammatory cells. TGFβ may also be produced endogenously by the MV.

Emerging evidence suggests that EndMT is also involved in tissue injury leading to tissue fibrosis is fibrosis diseases. For example, EndMT is associated with progressive fibrosis in kidney disease. While fibroblasts are not particularly abundant in the normal kidney, there is a marked increase in the number of fibroblasts at the onset of fibrogenesis. Furthermore, EndMT also contributes to the fibrotic responses observed in several lung pathologies, such as rejecting lung allografts, silica-induced lung carcinogenesis, and in idiopathic pulmonary fibrosis.

Other examples of pathological EndMT remodeling and fibrosis that lead to organ and tissue failure are found in cerebral cavernous malformations (Maddaluno et al, Nature 2013, 498:492-6), arterial calcification (Yao et al, Circ Res 2013, 113:495-504), and intimal thickening (Cooley et al, Science Trans Med, 2014, 6(227): 227ra34; and Chen P Y et al, Science Signaling 2014, 7(344):ra90).

### SUMMARY

### Abbreviations Used in this Disclosure

CD45 or PTPRC=protein tyrosine phosphatase, receptor type, C. VE-cad=VE-cadherin. This is an endothelial cell biomarker. SMA or α-SMA=alpha smooth muscle actin or smooth muscle alpha actin. This cytoskeletal protein is increased during EndMT but also in myofibroblasts and smooth muscle cells. Slug or Snai2=a transcription factor active during EndMT. MMP2=matrix metalloproteinase-2. NFATc1=nuclear factor in activated T cells. This is a transcription factor required for heart valve development; is expressed in endothelial cells that do not undergo EndMT. Therefore, when EndMT occurs, NFATc1 decreases with EndMT. Col 1=collagen 1. Col 3=collagen 3. TGF-β=transforming growth factor β. TGF beta1-3=transforming growth factor β 1, 2 and 3. ECs=endothelial cells. MCs=mesenchymal cells. EndMT=endothelial-to-mesenchymal transition. MI=myocardial infarction. IMI=inferior myocardial infarction. MV=mitral valve. VEC=valve endothelial cell. VIC=valve interstitial cell. MR=mitral regurgitation. CAEC=carotid artery endothelial cell. FBS=fetal bovine serum. PTP or PTPase=protein tyrosine phosphatase. ESA=endocardial surface area. VCW=vena contracta width.

Embodiments of the present disclosure are based on the discovery that mitral VECs can express CD45, a protein tyrosine phosphatase, both during in vivo post-myocardial infarction (MI) and in in vitro in response to exposure to TGFβ1. Specifically, FIG. 12A-12G show a correlation of CD45+ cells with fibrosis in the mitral leaflets after myocardial infarction and with infarct size. More specifically, increases in non-hematopoietic CD45+ cells correlate with severity of mitral regurgitation and extent of left ventricular remodeling and negatively correlate with ejection fraction. Besides mitral VECs, the inventors also showed that artificially activating endogenous CD45 expression using a CRISPR/Cas9 strategy in human endothelial cells led to CD45 expression and the induction of a common EndMT marker α-smooth muscle actin (αSMA). This observation indicates that CD45 is sufficient to drive EndMT in human endothelial cells, not just mitral VECs. The involvement of CD45+ endothelial population in the pathological EndMT remodeling and fibrosis during mitral valve (MV) adaptation and fibrosis post-MI provides a target for mitigating pathological endothelial-to-mesenchymal transition (EndMT), specifically blocking or inhibiting pathological EndMT, and for the treatment, prevention, and/or management of any medical conditions that comprises pathological EndMT.

Accordingly, this disclosure provide strategies and compositions for blocking or inhibiting pathological EndMT, thereby providing a treatment, prevention, or management of medical conditions that comprise pathological EndMT during the course of the medical conditions.

In one embodiment, provided herein is an inhibitor of CD45 for use in the inhibition of pathological EndMT in an endothelial cell. In another embodiment, provided herein is an inhibitor of CD45 for use in the manufacture of medicament for the inhibition of pathological EndMT in an endothelial cell.

In one embodiment, provided herein is an inhibitor of CD45 for use in the inhibition of pathological EndMT in a mammal. In another embodiment, provided herein is an inhibitor of CD45 for use in the manufacture of medicament for the inhibition of pathological EndMT in a mammal.

In one embodiment, provided herein is an inhibitor of CD45 for use in the prevention, treatment or management of a medical condition that involved pathological EndMT.

In one embodiment, provided herein is an inhibitor of CD45 for use in the manufacture of medicament for the prevention, treatment or management of a medical condition that involved pathological EndMT.

In one embodiment, the CD45 inhibitor is used in conjunction with a TGF-β inhibitor.

In another embodiment, the CD45 inhibitor is used in conjunction with an anti-fibrosis agent, e.g., human recombinant decorin. The anti-fibrosis agent is not a CD45 inhibitor or a TGF-beta inhibitor.

Additionally, in one embodiment, this disclosure provides a composition comprising a CD45 inhibitor. Additionally, the composition further comprises an inhibitor of TGF-beta, an inhibitor of tissue fibrosis wherein the inhibitor is not a CD45 inhibitor or a TGF-beta inhibitor, a pharmaceutically acceptable carrier, or various combinations of a TGF-beta inhibitor, a tissue fibrosis inhibitor and a pharmaceutically acceptable carrier.

Accordingly, in another embodiment, this disclosure provides a composition comprising a CD45 inhibitor and an inhibitor of TGF-beta. In one embodiment of this composition, the composition further comprises a pharmaceutically acceptable carrier.

In another embodiment, this disclosure provides a composition comprising a CD45 inhibitor, an inhibitor of TGF-beta and an inhibitor of tissue fibrosis wherein the inhibitor is not a CD45 inhibitor or a TGF-beta inhibitor. In one embodiment of this composition, the composition further comprises a pharmaceutically acceptable carrier.

In one embodiment, provided herein is a composition comprising a CD45 inhibitor for use in the inhibition of EndMT in an endothelial cell.

In one embodiment, provided herein is a composition comprising a CD45 inhibitor for use in the inhibition of EndMT in a mammal.

In one embodiment, provided herein is a composition comprising an inhibitor of CD45 and an inhibitor of TGF-beta for use in the inhibition of EndMT in an endothelial cell.

In one embodiment, provided herein is a composition comprising an inhibitor of CD45 and an inhibitor of TGF-beta for use in the inhibition of EndMT in a mammal.

In one embodiment of a composition described, the composition further comprises a pharmaceutically acceptable carrier.

In one embodiment of a composition described, the composition further comprises an inhibitor of TGF-beta.

In one embodiment of a composition described, the composition further comprises an inhibitor of tissue fibrosis, e.g., human recombinant decorin. The inhibitor is not a CD45 inhibitor or a TGF-beta inhibitor.

In one embodiment, provided herein is a pharmaceutical composition comprising an inhibitor of CD45 and a pharmaceutically acceptable carrier for use in the inhibition of EndMT in an endothelial cell.

In one embodiment, provided herein is a pharmaceutical composition comprising an inhibitor of CD45 and a pharmaceutically acceptable carrier for use in the inhibition of EndMT in a mammal.

In one embodiment, provided herein is a pharmaceutical composition comprising an inhibitor of CD45, an inhibitor of TGF-beta, and a pharmaceutically acceptable carrier for use in the inhibition of EndMT in an endothelial cell.

In one embodiment, provided herein is a pharmaceutical composition comprising an inhibitor of CD45, an inhibitor of TGF-beta, and a pharmaceutically acceptable carrier for use in the inhibition of EndMT in a mammal.

In one embodiment of a pharmaceutical composition described, the pharmaceutical composition further comprises an inhibitor of tissue fibrosis, e.g., human recombinant decorin. The inhibitor is not a CD45 inhibitor or a TGF-beta inhibitor.

Additionally, in one embodiment, this disclosure provides a method of inhibiting pathological EndMT comprising contacting an endothelial cell with an inhibitor of protein tyrosine phosphatase, receptor type, C, (PTPRC or CD45), that is, an inhibitor of CD45.

In another embodiment, this disclosure provides a method of inhibiting a pathological EndMT in a mammal comprising administering an effective amount of an inhibitor of CD45.

In another embodiment, this disclosure provides a method of treatment or management of a medical condition that comprise pathological EndMT during the course of the medical condition in a mammal comprising administering an effective amount of an inhibitor of CD45.

In another embodiment, this disclosure provides a method of preventing pathological EndMT after a disease or an injury comprising administering an effective amount of an inhibitor of CD45.

In one embodiment of any one of the disclosed methods, the pathological EndMT occurs in a disease or injury. For example, after MI.

In one embodiment of any one of the disclosed methods, the pathological EndMT produces excessive or undesirable tissue remodeling.

In one embodiment of any one of the disclosed methods, the pathological EndMT produces excessive or undesirable fibrosis.

In one embodiment of any one of the disclosed methods, the pathological EndMT occurs in conditions selected from the group consisting of mitral regurgitation (MR), myocardial infarction (MI), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, hepatic fibrosis, cirrhosis, fibrosis induced transplant antibody mediated rejection, cerebral cavernous malformations, arteriosclerosis, intimal hyperplasia in vein-to-arterial graft and pulmonary fibrosis.

In one embodiment of any one of the disclosed methods, the method further comprising detecting the expression of CD45 in the endothelial cells.

In one embodiment of any one of the disclosed methods, the method further comprising detecting the presence of EndMT.

In one embodiment of any one of the disclosed methods, the detection of EndMT comprises assessing expression of at least one biomarker, for examples, Fascin1 (FSCN1), αSMA, Fsp1, fibronect, Col 1, Col 3, vimentin and Hsp47.

In one embodiment of any one of the disclosed methods, the CD45 inhibitor is targeted to an endothelial cell. For examples, by targeting adhesion molecules expressed on endothelial cells, such as selectins, VCAM-1, PECAM-1, and ICAM-1. In one embodiment, the endothelial cell is undergoing EndMT.

In one embodiment of any one of the disclosed methods, the CD45 inhibitor is targeted to endothelial cells expressing CD45.

### BRIEF DESCRIPTION OF THE DRAWINGS

FIG. 1A-1D show the CD45 expression in mitral valves (MVs). Ovine MV leaflets from animals 6 months after IMI (FIGS. 1A, 1B, 1D) and control animals (FIG. 1C) analyzed for CD45 by immunohistochemistry. IgG staining served as a negative control (FIG. 1D).

FIG. 2 shows the CD45/VE-Cadherin/αSMA analysis of MV cells 6 months after inferior MI (IMI). A representative flow cytometric analysis is shown. Top panels show the labeling for VE-cadherin, αSMA and CD45. VE-Cadherin-positive/CD45-positive cells were analyzed in the APC-channel for αSMA-positive cells. This shows the distribution of VE-cadherin-positive/CD45-positive cells into αSMA-negative and αSMA-positive subpopulations. Bottom row shows cells labeled with isotype-matched IgGs to establish background staining.

FIG. 3A-3E show the flow cytometry of mitral VEC clones and non-valvular CAEC. Mitral VEC clone E10 (FIGS. 3A and 3B) and CAECs (FIGS. 3C and 3D) treated with EBM-B, (FIGS. 3A and 3C) or EBM-B+1 ng/ml TGFβ1 (FIGS. 3B and 3D) for 96 hours to induce EndMT. (FIG. 3E) Four additional mitral VEC clones treated with EBM-B+1 ng/ml TGFβ1 for 96 hours to show range of CD45 induction. Clones E5, D1, 14 and C5 were analyzed for VE-cadherin and CD45.

FIG. 4 shows the bar graphs of the summary of flow cytometric analysis of mitral valves from FIG. 3 and Table 1. The bar graphs combines CD45+ populations into VEC and non-endothelial.

FIG. 5: Mitral VEC clones show pure EC phenotype.

FIG. 6 shows the design of cell culture assay for effects of CD45 inhibitor on cell migration of EndMT increased migration.

FIG. 7A shows that CD45 phosphatase inhibitor blocks EndMT-increased migration.

FIG. 7B shows that CD45 phosphatase inhibitor has no effect on carotid artery endothelial cells (CAEC) that do not undergo EndMT and do not express CD45.

FIG. 8 shows that CD45 phosphatase inhibitor reduces EndMT and fibrosis markers in mitral VEC (e.g. Col1, Col3 and TGFbeta1-3).

FIG. 9 shows that TGF-beta does not induced EndMT in CAEC, and that the CD45 phosphatase inhibitor had no effect in CAEC not experiencing EndMT.

FIGS. 10A and 10B show that the changes in expression of CD45, VE-cadherin and αSMA upon exposure to TGFβ1. The expression levels were measured as mRNA levels in mitral VEC clones and CAEC by qPCR. Cells were treated for 4 days without (gray bars, control) or with (black bars) TGFβ1.

FIGS. 11A and 11B show that increased EndMT migration can be blocked by CD45 PTPase inhibitor. Mitral VECs (FIG. 11A) and CAEC (FIG. 11B) were treated without (light gray bars) or with TGFβ1 (1 ng/mL) for 4 days (dark gray bars) to induce EndMT. Cells were treated ±CD45 PTPase (1.0 μM) for 30 minutes prior to and during the migration assay, as indicated. Cells were allowed to migrate across Transwell membranes towards either endothelial basal media alone or endothelial basal media+ serum and bFGF for 6 hours.

FIG. 12A-12G collectively show that fibrosis, MR severity, infarct size and LV remodeling correlate with CD45+ cells.

FIG. 12A are representative images of Masson Trichrome-stained MV leaflets from sham (n=5) and IMI-6 month sheep.

FIGS. 12B and 12C are histograms showing the quantification of positively stained areas of collagen (Masson Trichrome) (FIG. 12B) and anti-CD45 in adjacent sections (FIG. 12C). Data were analyzed by Student's t-test using GraphPad Prism v7.0; p<0.05 considered significant.

FIG. 12D shows the positive correlation of total CD45+ cells with infarct heart size. Total CD45+ cells, measured by flow cytometry, in individual IMI-6 month sheep were plotted against infarct size (normalizing for heart size as infarct relative to total LV endocardial surface area).

FIG. 12E shows the positive correlation of CD45+ cells with VCW. MR was determined by measuring the width of the proximal jet (vena contracta) in the apical long-axis view. Values for individual sheep in sham (x) and IMI-6 (▴) groups were plotted against CD45+ cells (VE-cadherin+/CD45+/αSMA+, VE-cadherin+/CD45+/αSMA−, and VE-cadherin−/CD45+/αSMA+).

FIG. 12F shows the positive correlation of CD45+ cells with LV remodeling. LV remodeling, determined by the ratio of infarct endocardial surface area (ESA) at time of sacrifice (T2)/ESA at 30 minutes post-MI (T1), was plotted against the same CD45+ cells as in FIG. 12E.

FIG. 12G shows the ejection fraction plotted against the same CD45+ cells as FIG. 12E and FIG. 12F. The figure shows the negative correlation of CD45+ cells with ventricular ejection fraction. FIG. 12D-12G, Linear regression analysis was performed and the R² value was calculated to see how well the regression line fit the data. P<0.05 was considered significant.

### DETAILED DESCRIPTION

Unless otherwise explained, all technical and scientific terms used herein have the same meaning as commonly understood by one of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Definitions of common terms in molecular biology can be found in The Merck Manual of Diagnosis and Therapy, 19th Edition, or the 2015 digital online edition at merckmanuals.com; Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties. Further, unless otherwise required by context, singular terms shall include pluralities and plural terms shall include the singular.

Unless otherwise stated, the present disclosure was performed using standard procedures known to one skilled in the art, for example, in Michael R. Green and Joseph Sambrook, Molecular Cloning: A Laboratory Manual, Cold Spring Harbor Laboratory Press, Cold Spring Harbor, N.Y., USA (2012); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Meolcular Biology, Vol. 203, 2003, Transgenic Mouse, editored by Marten H. Hofker and Jan van Deursen, which are all herein incorporated by reference in their entireties.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Embodiments of the present disclosure are based on the discovery that mitral VECs can express CD45, a protein tyrosine phosphatase, both during in vivo post-myocardial infarction (MI) and in in vitro in response to TGF-β1. The inventors discovered that CD45 is expressed in mitral valve endothelial cells undergoing EndMT. The inventors showed that CD45 phosphatase inhibitor blocks EndMT-increased migration but the CD45 phosphatase inhibitor has no effects on carotid artery endothelial cells (CAEC) that do not undergo EndMT and do not express CD45.

The contribution of this CD45+ endothelial population to mitral valve (MV) adaptation and fibrosis post-MI during pathological EndMT remodeling and fibrosis provides a target for mitigating pathological EndMT, specifically blocking or inhibiting pathological EndMT, and for the treatment, prevention, and/or management of any medical conditions that comprises pathological EndMT.

Besides mitral VECs, the inventors also showed that by artificially activating endogenous CD45 expression in human endothelial cells using a CRISPR/Cas9 strategy, a good amount of CD45 expression was induced together with a committant induction of a common EndMT marker α-smooth muscle actin (αSMA). This observation indicates that the expression or presence of CD45 is sufficient to drive EndMT process in human endothelial cells, not just mitral VECs.

Accordingly, in one embodiment, this disclosure provides a method of inhibiting pathological EndMT comprising contacting an endothelial cell with an inhibitor of protein tyrosine phosphatase, receptor type, C, (PTPRC or CD45), that is, an inhibitor of CD45.

In another embodiment, this disclosure provides a method of inhibiting a pathological EndMT in a mammal comprising administering a therapeutically effective amount of an inhibitor of CD45.

In another embodiment, this disclosure provides a method of treatment or management of a medical condition that comprise pathological EndMT during the course of the medical condition in a mammal comprising administering a therapeutically effective amount of an inhibitor of CD45.

In another embodiment, this disclosure provides a method of preventing pathological EndMT after a disease or an injury comprising administering a therapeutically effective amount of an inhibitor of CD45.

An “effective amount” as the term is used herein, is used to refer to an amount that is sufficient to produce at least a reproducibly detectable amount of the desired results. In the context of the present disclosure, in some embodiments, effective amounts are amounts that inhibit CD45 or TGF-β activity or expression as described herein. In one embodiment, effective amounts are amounts that inhibit EndMT, as determined by EndMT biomarkers. Meaning a reduction of EndMT biomarkers in the presence of an relevant inhibitor compared to controls that are in the absence of the same inhibitor. One example of an effective amount is an amount that results in substantial inhibition of CD45 or TGF-β activity or expression in the endothelial cells contacted or in the treated mammal, or substantial reduction of EndMT compared to control. Substantial inhibition may comprise inhibition of greater than 5%, 10%, 20%, 30%, 40%, 50%, 60%, 70%, 80%, or 90% of detectable activity such as that in an identically treated control that is not exposed to the respective inhibitor. Such inhibition can be measured directly or indirectly. Direct measurement involves identification of binding, phosphatase activity, cell signaling events, EndMT biomarker expression, or receptor binding, or other direct measurements of CD45 or TGF-β activity such as cell signalling or cell adhesion function. Indirect measurement involves quantitation of overall EndMT activity, or other measurements of CD45 or TGF-β activity such as the assays provided herein. An effective amount will vary with the specific conditions and circumstances. Such an amount can be determined by the skilled practitioner for a given situation. The effective amounts can be provided all at once in a single administration or in fractional amounts that provide the effective amount in several administrations.

The term “therapeutically effective amount” refers to an amount that is sufficient to effect a therapeutically significant reduction in one or more symptoms of the condition when administered to a typical subject who has the condition. In one embodiment, the term “therapeutically effect” is used herein in a broad sense and includes prophylactic effects. A therapeutically significant reduction in a symptom or complication resulting from pathological EndMT or fibrosis is, e.g. about 10%, about 20%, about 30%, about 40%, about 50%, about 60%, about 70%, about 80%, about 90%, about 100%, or more (e.g., 1.5 fold, 2 fold, 3 fold, 4 fold, 5 fold, 10 fold, 25 fold, 50 fold, 100 fold, etc.) as compared to a control or non-treated subject. The term “therapeutically effective amount” refers to the amount of an agent determined to produce any therapeutic response in a subject. For example, a therapeutically effective amount of an inhibitor of CD45 or TGF-β may decrease the amount of EndMT biomarkers expressed over time as compared to a similar mammal who has not received the inhibitor.

Treatments that are therapeutically effective within the meaning of the term as used herein, include treatments that reduce or eliminate pathological EndMT discussed herein. For example, a treatment comprising a CD45 inhibitor can lead to a reduction or decreased in the expression of CD45, or the biomarkers that indicate EndMT or a delay in MV dysfunction after MI, delay in cerebral cavernous malformations, arterial calcification, intimal thickening, or a delay in organ or tissue failure. For example, a reduction or decreased of the expression of CD45, α-SMA, Slug, MMP2, NFATc1, TGFβ2, collagen 1, collagen 3, Fascin1, vimentin, and Hsp47 by at least 10%, at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99%, and by at least 100% compared to a respective control reference in the absence of the CD45 inhibitor. Such therapeutically effective amounts are readily ascertained by one of ordinary skill in the art. Thus, to “treat” means to deliver such an amount.

The precise determination of what would be considered a therapeutically effective amount may be based on factors individual to each subject, including their size, age, injury, and/or disease or injury being treated, and amount of time since the injury occurred or the disease began. One skilled in the art will be able to determine the therapeutically effective amount for a given subject based on these considerations which are routine in the art.

The term “treat” or “treatment” refers to therapeutic treatment wherein the object is to eliminate or lessen symptoms. Beneficial or desired clinical results include, but are not limited to, elimination of symptoms, alleviation of symptoms, diminishment of extent of condition, stabilized (i.e., not worsening) state of condition, delay or slowing of progression of the condition.

The inventors analyzed endothelial, interstitial and hematopoietic cells in MVs from normal and post-MI sheep to assess and quantify cellular changes in the MV post-MI as these might contribute to fibrosis.

The inventors discovered that CD45 is expressed in mitral valve endothelial cells undergoing EndMT. CD45 is a phosphatase, and it is best known as a pan-hematopoietic cell marker. The CD45 is detected in vivo in mitral and aortic valves and can be induced in vitro in valve endothelial cells by TGF-β, which is a strong stimulator of EndMT. Endothelial cells (ECs) that do not undergo TGFβ-mediated EndMT, such as normal healthy CAEC, do not express CD45 (±TGFβ). Therefore, the discovery herein puts CD45 expression and EndMT occurrence together at the same time and at the same location.

The inventors showed for the first time CD45 immunostaining along the mitral valve endothelium in an ovine model of inferior myocardial infarction (IMI). This provided the first clue that CD45 might be expressed in valve endothelial cells post-myocardial infarction, in response to the cytokine storm caused by the ischemia.

The inventors further tested the requirement for CD45 in EndMT using a commercially available inhibitor of the CD45 phosphatase activity. The results showed the following: (1) the CD45 phosphatase inhibitor specifically blocks TGFβ-induced migration of mitral valve endothelial cells; please note: increased cellular migration is a hallmark of EndMT; (2) The CD45 phosphatase inhibitor blocks the induction of 7 EndMT/fibrosis markers (α-SMA, Slug, MMP2, NFATc1, TGFβ2, collagen 1 and collagen 3; (3) It was also noted that NFATc1 is decreased in upon the onset and progression of EndMT; and (4) CD45 phosphatase inhibitor blocks the decrease of NFATc1.

The experiments disclosed herein showed CD45 is expressed in valve endothelial cells induced to undergo endothelial to mesenchymal transition (EMT or EndMT) but not in endothelial cells unable to undergo TGF-β-induced EndMT.

Besides mitral VECs, the inventors also showed that by artificially activating endogenous CD45 expression in human endothelial cells using a CRISPR/Cas9 strategy, a good amount of CD45 expression was induced together with a committant induction of a common EndMT marker α-smooth muscle actin (αSMA). This observation indicates that the expression or presence of CD45 is sufficient to drive EndMT process in human endothelial cells, not just mitral VECs.

In one embodiment of a disclosed method or composition described, the pathological EndMT occurs in a disease or injury. Non limiting examples where pathological EndMT can occur following a disease, infection or injury include MR occurring after MI, cancer, fibrodysplasia ossificans progressive (FOP), systemic sclerosis, hypertension, diabetes mellitus-induced cardiac fibrosis, renal fibrosis, hepatic fibrosis, cirrhosis, keloid formation, fibrosis induced transplant antibody mediated rejection, lung infection, kidney infection, liver infection (hepatitis infection), alcohol abuse, or anti-freeze poisoning etc.

In one embodiment of a disclosed method or composition described, the pathological EndMT produces excessive or undesirable tissue remodeling. Non limiting examples where pathological EndMT can occur as a result of excessive or undesirable tissue remodeling include systemic sclerosis, renal fibrosis, cirrhosis, keloid formation, intimal hyperplasia in vein-to-arterial graft and pulmonary fibrosis etc.

In one embodiment of a disclosed method or composition described, the pathological EndMT occurs in conditions selected from the group consisting of mitral regurgitation (MR), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, fibrosis induced transplant antibody mediated rejection, cerebral cavernous malformations, arteriosclerosis, intimal hyperplasia in vein-to-arterial graft and pulmonary fibrosis. Pathological EndMT also occurs during the implantation of various tissue grafts. Non-limiting examples include renal allograft, a heart allograft, a lung allograft, a liver allograft, a pancreas allograft, an intestine allograft, a body member allograft, a muscle allograft, and a face engraft.

As used herein the term “allograft” refers to a surgical transplant of tissue between genetically different individuals of the same species. The term allograft excludes isografts and xenografts. The term allograft encompasses any solid organ transplant such as a renal allograft, a heart allograft, a lung allograft, a liver allograft, a pancreas allograft, an intestine allograft and/or a body member allograft (e.g. hand, feet, leg, etc). It also includes other allografts such as muscle allograft, face engraft, eyes, etc. In one embodiment of a disclosed method or composition described, the method further comprises identifying a mammal having a medical condition comprising pathological EndMT.

In one embodiment of a disclosed method or composition described, the method further comprises selecting for a mammal having a medical condition comprising pathological EndMT. For examples, the selection step comprises detecting the expression of CD45 in the endothelial cells or detecting the presence of EndMT according to any methods known in the art. For example, as described herein in the example section and description below.

In one embodiment of a disclosed method or composition described, the method further comprises selecting for a mammal having a medical conditions selected from the group consisting of mitral regurgitation (MR), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, fibrosis induced transplant antibody mediated rejection, cerebral cavernous malformations, arteriosclerosis, intimal hyperplasia in vein-to-arterial graft and pulmonary fibrosis.

In one embodiment of a disclosed method or composition described, the mammal is a primate mammal. In one embodiment of any of disclosed methods, the primate mammal is a human.

In one embodiment of a disclosed method or composition described, the method further comprising detecting the expression of CD45 in the endothelial cells or in a biological sample from the mammal. For example, a biopsy of the tissue that has sustained an injury (e.g., MI, lung infection, kidney infection, allograft etc.) can be taken and the expression of CD45 in the endothelial cells therein can be analyzed. In another embodiment, a biopsy of the tissue in the disease condition, (e.g., FOP, SSc, pulmonary fibrosis etc.) can be taken and the expression of CD45 in the endothelial cells therein can be analyzed. A skilled artisan can use any methods known in the art to determine the expression of CD45 in the endothelial cells. For example, as described in the Example section of this disclosure, by immunostaining or by Western Blotting analysis. In one embodiment, an increased expression level for CD45 when compared to a control value is indicative of the presence or likelihood of EndMT, and therefore treatment with a CD45 inhibitor is warranted to prevent or inhibit further undesired pathologic EndMT. In one embodiment, the biopsy sample for detecting the expression of CD45 is a biological sample, such as a tissue sample, a biopsy of an allograft, a urine sample or a plasma sample. In one embodiment, the control CD45 value is the CD45 expression level obtained for a healthy mammal in the absence of a disease or injury or allograft, the CD45 expression level is obtained from a corresponding biological sample. Meaning, if the test biological sample is a urine sample, the control CD45 value is that obtained from a urine sample of a healthy mammal.

In one embodiment of disclosed method or composition described, the method further comprising detecting the presence of EndMT. Biomarkers associated with EndMT are known in the art. For examples, the EndMT/fibrosis markers disclosed herein, and Fascin1, Vimentin and Hsp47, as described in the International Patent PCT publication WO 2013150375 and US patent application publication No.: US 2015/0118224, the contents of both publications are incorporated herein by reference in their entirety. The expression levels of these biomarkers of EndMT can be determined or measured by any method known in the art, for example, by immunostaining, by Western Blotting analysis, or by quantitative reverse transcription polymerase chain reaction (qRT-PCR). In one embodiment, an increased in the expression level for each of Fascin1, Vimentin and Hsp47 when compared to a control value is indicative of the presence of EndMT. In one embodiment, an increased expression level for any one of Fascin1, Vimentin and Hsp47 when compared to a respective control value is indicative of the presence or likelihood of EndMT, and therefore treatment with a CD45 inhibitor is warranted to prevent or inhibit further undesired pathologic EndMT. In one embodiment, the control value for the EndMT biomarkers Fascin1, Vimentin and Hsp47 is the respective expression level obtained for a healthy mammal in the absence of a disease or injury or allograft. In one embodiment, the detection of the presence of EndMT is performed via a biological sample. For example, the biological sample is a biopsy of the allograft. In another embodiment, the biological sample is a urine sample or a plasma sample.

By “increase” in the expression level of the disclosed biomarkers in the endothelial cells or the biological sample means at least 5% higher, at least 8% higher, at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than a comparable control cells or biological sample obtain from a healthy subject who is not experiencing a disease or injury or had an allograft.

By “decrease” or “reduce in the expression level of the disclosed biomarkers in the endothelial cells or the biological sample means at by at least 10%, at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 99%, and by at least 100% compared to a control reference in the absence of a disclosed inhibitor.

In one embodiment of a disclosed method or composition described, the detection of the presence of EndMT comprises obtaining a biological sample such as a biopsy of the allograft, a biopsy of tissue that has sustained an injury, a urine sample or a plasma sample for determining the level of CD45 or the levels of the EndMT biomarkers Fascin1, Vimentin and Hsp47.

In one embodiment of a disclosed method or composition described, the detection of the presence of EndMT comprises obtaining a biopsy sample of a tissue that has sustained an injury (e.g., MI, lung infection, kidney infection, etc.). The biopsy sample can be taken and analyzed for the expression of EndMT biomarkers in the endothelial cells.

In another embodiment of a disclosed method or composition described, the detection of the presence of EndMT comprises obtaining a biopsy sample of a tissue in the disease condition, (e.g., FOP, SSc, pulmonary fibrosis etc.). The biopsy sample can be taken and analyzed for the expression of EndMT biomarkers in the endothelial cells. A skilled artisan can use any methods known in the art to determine the expression of EndMT biomarkers in the endothelial cells. For example, as described in the WO 2013150375, and US patent application publication No.: US 2015/0118224, by immunostaining or by Western Blotting analysis or by reverse transcriptase polymerase chain reaction (RT-PCR).

In one embodiment of a disclosed method or composition described, the detection of EndMT comprises assessing expression of at least one biomarker selected from the group consisting of Fascin1, Vimentin and Hsp47.

In one embodiment of a disclosed method described, the method further comprises obtaining a biological sample from the mammal, e.g., a human patient; and detecting the presence of EndMT, e.g., via detecting an increased expression of Fascin1, or Vimentin, or Hsp47, prior administering a CD45 inhibitor or a composition comprising a CD45 inhibitor described herein.

In one embodiment of a disclosed method described, the method further comprises obtaining a biological sample from the mammal, e.g., a human patient; and detecting the presence of EndMT, e.g., via detecting an increased expression of CD45, prior administering a CD45 inhibitor or a composition comprising a CD45 inhibitor described herein.

In one embodiment of a disclosed method or composition described, the CD45 inhibitor is targeted to an endothelial cell. For examples, by targeting adhesion molecules expressed on endothelial cells, such as selectins, VCAM-1, PECAM-1, and ICAM-1. In one embodiment, the endothelial cell is undergoing EndMT. Methods of drug delivery targeting endothelial cells are known in the art. For examples, drugs are conjugated with binding ligands of adhesion molecules expressed on endothelial cells are described in U.S. Pat. No. 7,182,933, U.S. Pat. No. 7,479,483, US20020044959, and WO2015108783, the contents of these publications are incorporated herein by reference in their entirety.

In one embodiment of a disclosed method or composition described, the CD45 inhibitor is targeted to endothelial cells expressing CD45.

It is also envisioned that the methods and compositions described herein can be used as prophylaxis.

In one embodiment of a disclosed method or composition described, the method further comprises administering an inhibitor of TGF-beta (TGF-β). In some embodiments, the inhibitor of TGF-3 is a chemical compound, a small molecule inhibitor, an antibody against TGF-β or fragment thereof, or a nucleic acid inhibitor of TGF-β. In one embodiment, the nucleic acid inhibitor of TGF-β inhibits the expression of TGF-β.

In one embodiment of a disclosed method or composition described, the CD45 inhibitor and the inhibitor of TGF-β are contacted with the endothelial cell or administered simultaneously or concurrently to the mammal.

In another embodiment of a disclosed method or composition described, the CD45 inhibitor and the inhibitor of TGF-beta are contacted with the endothelial cell or administered sequentially to the mammal.

### Endothelial to Mesenchymal Transition (EndMT) and Pathological EndMT

Endothelial to mesenchymal transition (EndMT) is a normal process that occurs during development and probably occurs at a low level throughout life. During this process, the endothelial cells convert to a more mesenchymal cell type that can give rise to cells such as fibroblasts and bone cells. EndMT is essential during embryonic development and tissue regeneration. Interestingly, it also plays a role in pathological conditions like fibrosis of organs such as the heart and kidney. For examples, EMT/EndMT is known to occur in disease settings, and appears to contribute to the vascular pathologies, such as in ischemic mitral regurgitation (IMR) after myocardial infarction (MI) (Dal-Bianco et al,⁵). IMR, a common complication after MI, induces adaptive cellular responses in the mitral valve (MV) that may be initially beneficial, but eventually lead to leaflet fibrosis and MV dysfunction. Other examples, beyond cardiac valves, are EndMT in cerebral cavernous malformations (Maddaluno et al, Nature 2013), arterial calcification (Yao et al, Circ Res 2013), and intimal thickening (Cooley et al, Science Trans Med, 2014; Chen P Y et al, Science Signaling 2014). In addition, EndMT contributes to the generation of cancer associated fibroblasts (CAF) that are known to influence the tumor-microenvironment favorable for the tumor cells. EndMT is a form of the more widely known and studied Epithelial-to-Mesenchymal Transition (EMT). Like EMT, EndMT can be induced by transforming growth factor (TGF)-β. Indeed many studies have pointed to the important role of TGF-β receptor/Smad signaling and downstream targets, such as Snail transcriptional repressor in EndMT. Now that CD45 expression is involved in EndMT, by selective blocking of EndMT and also the consequential signaling arising from the enzymatic activity of CD45, pathological EndMT may be inhibited for the therapeutic benefit of patients with cancer, pathological tissue remodeling and other fibrosis related diseases.

### Role of EndMT in Cancer

Fibroblasts are one the most abundant cell type in the microenvironment of tumors, being particularly prominent in carcinomas of colon, breast, pancreas, and prostate. There is substantial evidence that cancer-associated fibroblasts (CAFs) contribute to tumor growth and metastasis. This is mediated by the release of classical growth factors such as TGF-β, epidermal growth factor, hepatocyte growth factor, as well as a range of chemokines that influence diverse aspects of tumor cell behavior.

CAFs form a heterogeneous population, most likely related to their diverse origin. Whereas activation of local stromal fibroblasts has traditionally been considered the major source of CAFs, recently it was shown that EndMT is another unique source of CAFs. The EndMT in tumors was reported in two different mouse models of cancer and demonstrated that a substantial proportion of CAFs in these models arise through EndMT. The CAFs were shown to co-express the endothelial marker CD31 along with one of the mesenchymal markers, fibroblast specific protein (FSP)1, or α-smooth muscle actin (αSMA). Approximately, 40% of FSP1-positive CAFs were also found to be CD31 positive. To study the origin of the CAFs in more detail, tumors were grown in Tie2-Cre; R26R-lox-STOP-lox-lacZ transgenic mice, a reporter strain in which all cells of endothelial origin can be irreversibly labeled with lacZ. In 30% of the CAFs in tumors of these transgenic mice LacZ was detected making the authors conclude that the CAFs originated from endothelial cells. Since Tie2 is however also expressed in the hematopoietic lineage it is technically also possible that the lacZ positive CAFs originate from these cells.

These studies indicate that EndMT is an important mechanism for CAF recruitment to the tumour stroma. Since TGF-β signaling is a known mediator of EndMT and TGF-β is abundantly expressed in many different tumors, EndMT may be mediated by TGF-β produced in the tumor. Yet, the molecular mechanism of EndMT in tumors has not yet been specifically studied, but is to be expected to involve similar pathways as in fibrosis.

### EndMT in Tissue Fibrosis and Fibrosis Diseases

Fibrosis is the formation of excess fibrous connective tissue in an organ or tissue in a reparative or reactive process. This can be a reactive, benign, or pathological state. For example, in response to injury, this is called scarring, and if fibrosis arises from a single cell line, this is called a fibroma. Fibrosis is an essential process of proper wound healing. Physiologically, fibrosis acts to deposit connective tissue, which can obliterate the architecture and function of the underlying organ or tissue.

However, in many pathological conditions, fibrosis is the pathological state of excess deposition of fibrous tissue, as well as the process of connective tissue deposition in healing, that is, fibrosis is deregulated or excessive fibrosis occurs. The consequential effects of deregulated or excessive fibrosis include, but are not limited to, excessive tissue remodeling, lost tissue inflexibility, organ enlargement and/or failure, joint stiffness and loss of joint mobility and range of motion, and organ or vessel lumen obstruction or stenosis. Fibrosis can occur in many tissues within the body, typically as a result of inflammation or damage, and examples include: lungs-pulmonary fibrosis, e.g., cystic fibrosis and idiopathic pulmonary fibrosis (idiopathic meaning the cause is unknown); liver, e.g., cirrhosis; heart, e.g., atrial fibrosis, endomyocardial fibrosis, and myocardial infarction; arthrofibrosis (knee, shoulder, other joints); Crohn's Disease (resulting intestine fibrosis); Dupuytren's contracture (undesirable fibrosis in hands and fingers); keloid (excessive fibrosis in the skin); mediastinal fibrosis (excessive fibrosis in the soft tissue of the mediastinum); myelofibrosis (excessive fibrosis in the bone marrow); Peyronie's disease (excessive fibrosis or undesirable fibrosis in the penis); nephrogenic systemic fibrosis (skin); progressive massive fibrosis (in the lungs)—a complication of coal workers' pneumoconiosis; retroperitoneal fibrosis (excessive fibrosis or undesirable fibrosis in the soft tissue of the retroperitoneum); scleroderma/systemic sclerosis (excessive fibrosis or undesirable fibrosis in the skin and also the lungs); and adhesive capsulitis (excessive fibrosis or undesirable fibrosis in the shoulder).

The predominant cellular mediators of fibrosis are assumed to be (myo)fibroblasts, not only in heart fibrosis but also in fibrosis of organs such as lung, kidney, and the liver. Fibrosis of all these organs share similar pathways. The origin of these (myo)fibroblasts may, besides resident interstitial fibroblast, be cells derived from the bone marrow as well as fibroblastic cells that have transdifferentiated from cells of epithelial origin. More interestingly, these cells can also be derived from endothelial cells that have undergone EndMT.

In the kidney, for example, it was shown that EndMT can generate myofibroblasts in early diabetic renal fibrosis. Using endothelial-lineage tracing with Tie2-cre; LoxP-eGFP transgenic mice a significant number of interstitial α-smooth muscle actin-positive cells (myofibroblasts) were shown to be of endothelial origin in fibrotic kidneys from mice with streptozotocin-induced diabetic nephropathy. This indicated that EndMT can contribute to the early progression of diabetic nephropathy. Earlier it was already shown that fibroblasts expressed the endothelial marker CD31 in three different mouse models of renal disease: streptozotocin-induced diabetic nephropathy, unilateral ureteral obstructive nephropathy, and a mouse model of Alport syndrome (Zeisberg et al. 2008). Approximately 30% to 50% of fibroblasts formed in the kidneys of these models co-expressed the endothelial marker CD31 and the fibroblast/myofibroblast markers FSP1 and/or αSMA.

Likewise in the lung, fibrosis can cause serious pathological conditions such as idiopathic pulmonary fibrosis (IPF). IPF is characterized by progressive obliteration of normal alveolar lung architecture and replacement by fibrotic tissue. Scar formation, the accumulation of excess fibrous connective tissue, leads to thickening of the walls, and causes reduced oxygen supply in the blood. The result is declining lung function, progressive dyspnea, and ultimately death within 3 to 5 years of diagnosis. It has been demonstrated that pulmonary capillary endothelial cells, through EndMT, can serve as a source of fibroblasts in pulmonary fibrosis. This was shown by bleomycin-induced lung injury in mice where it was found, using lineage tracing methods, that the fibroblasts originated from the endothelial cells. Interestingly they also found a dependence on Ras for completion of EndMT. Treatment with TGF-β in combination with activated Ras induced a persistent morphological change and suppression of endothelial markers consistent with EndMT.

Systemic sclerosis (SSc) is a rare autoimmune disease of unknown cause characterized by diffuse fibrosis, degenerative changes, and vascular abnormalities in the skin, joints, and internal organs, where the dermal layer of the skin undergoes fibrosis. In addition systemic sclerosis results in inflammatory responses, vascular changes and loss of function of internal organs due to scarring and extracellular matrix deposition. Study recently demonstrated that EndMT could occur in abnormal fibrillin-1 expression and chronic oxidative stress mediated endothelial mesenchymal transition in a murine model of SSc, tight-skin (Tsk) (2/1) mice. This mesenchymal transition might contribute to the reduction in angiogenesis that is known to occur in this model of SSc.

### Endothelial-to-Mesenchymal Transition in Cardiac Fibrosis

In cardiac fibrosis the heart valves abnormally thicken due to inappropriate proliferation of cardiac fibroblasts and of disruption of normal myocardial structure through excessive deposition of extracellular matrix. Several studies have given evidence for the role of EndMT in cardiac fibrosis. Cardiac fibrosis was induced by exposing the heart to pressure overload for 5 days via aortic banding. Analysis of the fibrotic lesions revealed the presence of fibroblasts that originated from endothelial cells. The endothelial cells were shown to undergo EndMT and contribute to the total pool of cardiac fibroblasts, similar as in formation of the aterioventricular cushion in embryonic development.

Not only endothelial cells lining the vessel wall have been shown to be able to undergo EndMT. Also circulating endothelial progenitor cells have been shown to undergo EndMT. The conversion of these circulating endothelial progenitor cells to smooth muscle-like progeny was also shown to be stimulated by TGF-β.

Most cardiac diseases caused by inflammation are associated with fibrosis in the heart. Fibrosis is characterized by the accumulation of fibroblasts and excess deposition of extracellular matrix (ECM), which results in the distorted organ architecture and function. Recent studies revealed that cardiac fibroblasts are heterogeneous with multiple origins. Endothelial-mesenchymal transition (EndMT) plays important roles in the formation of cardiac fibroblasts during pathological settings.

Atrial fibrosis is a major contributor to the atrial fibrillation. EndMT describes the process of endothelial cell (EC) transformation into a myofibroblast—the major extracellular matrix synthesizing cell. endocardial EC layer may be an early sensor to increased atrial stress; endocardial EndMT may be an early event during the atrial remodeling process and perhaps contributes to global intra-atrial remodeling through altered paracrine and/or permeability mechanisms.

### EndMT Leading to Loss of Endothelial Function

As described above the result of EndMT in pathological conditions is fibrosis because of the generation of fibroblasts and excessive extracellular matrix. However EndMT also leads to the loss of endothelium. The loss of endothelial function can lead to badly perfused tissue and subsequent tissue damage. For example following traumatic spinal cord injury, significant vascular disruption occurs at the site(s) of injury. This interruption of vascular support is thought to be a key mediator of multiple secondary injury cascades, all of which contribute to loss of functional tissue.

### Endothelial Transdifferentiation is not Limited to Fibrosis

Other than differentiation towards myofibroblasts endothelial cells can also differentiate to other cell types.

Fibrodysplasia ossificans progressive (FOP) is a disorder in which muscle and connective tissues are slowly replaced by bone referred to as ossification. It is a severely debilitating disorder that is caused by an activating mutation in the BMP type I receptor, ALK2. In FOP-patients acute inflammation causes heterotopic ossification in soft tissues at nearly any site in the body. The source of the ossifying cells was previously unknown but Medici et al. showed that bone and cartilage cells from lesions of people with FOP and mice with mutant ALK2 expressed the endothelial markers Tie2 and von Willebrand factor. In lesions from mice with FOP induced by a transgene encoding constitutively active ALK2 with amino acid change Q207D, osteoblasts and chondrocytes expressed endothelial markers, suggesting that they arose from endothelial cells. Vascular endothelial cells infected with adenoviral constructs encoding R206H ALK2 can transform into multipotent mesenchymal stem-like cells, which can be triggered to differentiate into osteoblasts and chondrocytes. This suggests an endothelial origin of the ectopic mesenchymal cells that form the heterotopic tissues. Furthermore, human endothelial cells treated with BMP4 or TGF-β2, to activate endogenous ALK2, undergo EndMT resulting in mesenchymal stem cells. Thus, these data suggest that EndMT may contribute to the physiological process of fracture repair.

Cerebral cavernous malformation (CCM) is a vascular dysplasia, mainly localized within the brain and affecting up to 0.5% of the human population. CCM lesions are formed by enlarged and irregular blood vessels that often result in cerebral haemorrhages. CCM is caused by loss-of-function mutations in one of three genes, namely CCM1 (also known as KRIT1), CCM2 (OSM) and CCM3(PDCD10), and occurs in both sporadic and familial forms.

Recent studies have investigated the cause of vascular dysplasia and fragility in CCM, but the in vivo functions of this ternary complex remain unclear. Postnatal deletion of any of the three Ccm genes in mouse endothelium results in a severe phenotype, characterized by multiple brain vascular malformations that are markedly similar to human CCM lesions. Endothelial-tomesenchymal transition (EndMT) has been described in different pathologies, and it is defined as the acquisition of mesenchymaland stem-cell-like characteristics by the endothelium. It has been shown that endothelial-specific disruption of the Ccm1 gene in mice induces EndMT, which contributes to the development of vascular malformations. EndMT in CCM1-ablated endothelial cells is mediated by the upregulation of endogenous BMP6 that, in turn, activates the transforming growth factor-b (TGF-b) and bone morphogenetic protein (BMP) signalling pathway.

Veins grafted into an arterial environment undergo a complex vascular remodeling process. Pathologic vascular remodeling often results in stenosed or occluded conduit grafts. Understanding this complex process is important for improving the outcome of patients with coronary and peripheral artery disease undergoing surgical revascularization. Using in vivo murine cell lineage-tracing models, we show that endothelial-derived cells contribute to neointimal formation through endothelial-to-mesenchymal transition (EndMT), which is dependent on early activation of the Smad2/3-Slug signaling pathway. Antagonism of transforming growth factor-beta (TGF-β) signaling by TGF-beta neutralizing antibody, short hairpin RNA-mediated Smad3 or Smad2 knockdown, Smad3 haploinsufficiency, or endothelial cell-specific Smad2 deletion resulted in decreased EndMT and less neointimal formation compared to controls.

### CD45, the Leukocyte Common Antigen or Protein Tyrosine Phosphatase, Receptor Type, C

Protein tyrosine phosphatase, receptor type, C also known as PTPRC, is an enzyme that, in humans, is encoded by the PTPRC gene. PTPRC is also known as CD45 antigen (CD stands for cluster of differentiation), which was originally called leukocyte common antigen (LCA).

The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP contains an extracellular domain, a single transmembrane segment and two tandem intracytoplasmic catalytic domains, and thus belongs to receptor type PTP. The gene encoding for PTP is specifically expressed in hematopoietic cells. This PTP has been shown to be an essential regulator of T- and B-cell antigen receptor signaling. It functions through either direct interaction with components of the antigen receptor complexes or by activating various Src family kinases required for the antigen receptor signaling. This PTP also suppresses JAK kinases, and, thus, functions as a negative regulator of cytokine receptor signaling. Four alternatively spliced transcripts variants of this gene, which encode distinct isoforms, have been reported.

By “CD45” as used herein is meant a CD45 mRNA, protein, peptide, or polypeptide. The term “CD45” is also known in the art as PTPRC (protein tyrosine phosphatase, receptor type, C), B220, GP180, LCA, LY5, and T200. The sequence of human CD45 cDNA is recorded at GenBank Accession No. NM-002838.2. Other human CD45 sequences are recorded at GenBank Accession Nos. NM-080921.2, NM-080922.2, NM-080923.2, Y00062.1, Y00638.1, BC014239.2, BC017863.1, BC031525.1, BC121086.1, BC121087.1, BC127656.1, BC127657.1, AY429565.1, AY567999.1, AK130573.1, DA670254.1, DA948670.1, AY429566.1, and CR621867.1. Mouse CD45 mRNA sequences are found at GenBank Accession Nos. NM-011210.2, AK054056.1, AK088215.1, AK154893.1, AK171802.1, BC028512.1, EF101553.1, L36091.1, M11934.1, M14342.1, M14343.1, M15174.1, M17320.1, and M92933.1. Rhesus monkey CD45 mRNA sequence are found at GenBank Accession No. XR-012672.1.

The CD45 family consists of multiple members that are all products of a single complex gene. This gene contains 34 exons and three exons of the primary transcripts are alternatively spliced to generate up to eight different mature mRNAs and after translation eight different protein products. These three exons generate the RA, RB and RC isoforms.

Various isoforms of CD45 exist: CD45RA, CD45RB, CD45RC, CD45RAB, CD45RAC, CD45RBC, CD45RO, CD45R (ABC). CD45RA is located on naive T cells and CD45RO is located on memory T cells. CD45 is also highly glycosylated. CD45R is the longest protein and migrates at 200 kDa when isolated from T cells. B cells also express CD45R with heavier glycosylation, bringing the molecular weight to 220 kDa, hence the name B220; B cell isoform of 220 kDa. B220 expression is not restricted to B cells and can also be expressed on activated T cells, on a subset of dendritic cells and other antigen-presenting cells.

Naive T lymphocytes express large CD45 isoforms and are usually positive for CD45RA. Activated and memory T lymphocytes express the shortest CD45 isoform, CD45RO, which lacks RA, RB, and RC exons. This shortest isoform facilitates T cell activation.

The cytoplasmic domain of CD45 is one of the largest known and it has an intrinsic phosphatase activity that removes an inhibitory phosphate group on a tyrosine kinase called Lck (in T cells) or Lyn/Fyn/Lck (in B cells) and activates it.

The biological function of this glycoprotein remains to be resolved. In order to clarify the role of CD45 antigen in hematopoietic cell differentiation and function, its expression on human leukemia/lymphoma cell lines was studied by membrane immunofluorescence. Thirty-eight established cell lines were analyzed using T29/33, a monoclonal antibody (MoAb) that recognizes the common epitopes of this glycoprotein molecule. Conventional cell marker studies were also carried out on these cell lines to compare their CD45 expression. It was shown that CD45 expression varies among B-lineage cells depending on cell differentiation, in contrast to its stable expression on leukemic T cell (6/6, positive) and myeloid (5/5, positive) lineage cell lines. On the other hand, only two out of six histiomonocytoid lineage cell lines were positive. Human T cell leukemia/lymphoma virus type I (HTLV-I)-associated T cell lines derived from peripheral blood leukocytes of patients with adult T cell leukemia/lymphoma (ALT/L) in Japan did not express CD45 on their cell surface. Taken together, these observations suggest that CD45 has a functional role in hematopoietic cell activation and differentiation.

### Inhibitors of CD45 Tyrosine Phosphatase

As used herein, the term “CD45 inhibitor” refers to any inhibitor that blocks the phosphatase activity of CD45, blocks the cell signaling elicited by the protein CD45, or blocks the expression of CD45 in the cell. Encomposed within “CD45 inhibitors” are small molecule inhibitors, synthetic chemical compound inhibitors, peptide inhibitors, peptide mimetic inhibitors, antagonist of CD45 enzymatic activity, antagonist anti-CD45 antibodies and functional fragments therefrom, and nucleic acid inhibitors of CD45 gene expression.

In one embodiment, the CD45 inhibitor used in the disclosed methods or composition described is a chemical compound or a small molecule inhibitor. In another embodiment, the CD45 inhibitor used in the disclosed methods or composition described is a nucleic acid molecule. For example, an RNA or a DNA. In one embodiment, the nucleic acid molecule inhibits the expression of CD45. In some embodiment, the nucleic acid molecule is comprises modified nucleotides.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Chemical compounds that inhibit CD45 phosphatase activity are known in the art. Non-limiting examples include the potent reversible inhibitors of the protein tyrosine phosphatase CD45 such as 9,10-phenanthrenediones eg. N-(9,10-dioxophenanthren-2-yl)-2,2-dimethylpropanamide (PubChem CID: 9926586; SMILES: CC(C)(C)C(═O)NC1=CC2=C(C=C1)C3=CC═CC=C3C(═O)C2=O; CAS 345630-40-2); the various substituted phenanthrene-9,10-diones as described in U.S. Publication No.: US 2003/0207812, the contents are incorporated herein by reference in its entirety; the 4-(4-bromo-phenyl)-[1,2]naphthoquinone; 4-(3-nitro-phenyl)-[1,2]naphthoquinone; 5,6-dioxo-5,6-dihydro-naphthalene-1-carboxylic acid methyl ester, or 5,6-dioxo-5,6-dihydro-naphthalene-2-carboxylic acid methyl ester that are described in U.S. Pat. No. 6,939,896, the contents are incorporated herein by reference in its entirety; 1,4-naphthoquinone compounds e.g. Compounds 210 and 211 from Chembridge (San Diego, Calif.) Mol Pharmacol. 2014 April; 85(4):553-63; Pulchellalactamand (Wen-Ren Li et al. J. Org. Chem., 2002, 67 (14):4702-4706); and RWJ-60475-(AM)₃ is a cell-permeable acetoxymethyl (AM) ester of RWJ-60475. FORMULA: C₂₂H2₃BrNO₁₃P, (catalog # BML-PR110-0001) of ENZO® LifeSciences Inc.

Other exemplary inhibitors of CD45 include those described in U.S. Pat. No. 6,939,896; WO2001/045681, and U.S. Publication No.: US2002/0168362, US2003/0119897 US2003/0130298, US 2003/020781, and US 2014/0079725, the contents of each which are herein incorporated by reference in their entirety.

Nucleic acid based CD45 inhibitor are described in U.S. Pat. Nos. 8,288,525 and 8,912,316, and in U.S. Publication No.: US2011/0034537, US2013/0065943, and US2015/0093417, the contents of which are incorporated herein by reference in their entirety. For examples, cuGGcuGAAuuucAGAGcATsT (SEQ. ID. NO: 1) and UGCUCUGAAAUUcAGCcAGTsT (SEQ. ID. NO: 2) described in US2013/0065943. In one embodiment, the nucleic acid based CD45 inhibitor forms dsRNA and targets the cleavage of a CD45 mRNA. In one embodiment, the nucleic acid based CD45 inhibitor inhibits the expression of CD45. In another embodiment, the nucleic acid based CD45 inhibitor inhibits a CD45 mRNA and cause RNA interference. For example, as described in T. I. Novobrantseva et al., Molecular Therapy Nucleic Acids (2012), 1:e4.

Anti-CD45 leukocyte antigen antibodies are also known in the art. For examples, as described in U.S. Pat. No. 6,106,834, and U.S. Publication No.:US20020168362, the contents of each which are herein incorporated by reference in their entirety.

In another embodiment, the CD45 inhibitor is an anti-CD45 antibody or fragment thereof that binds and inhibits the activity of the CD45.

In one embodiment, the anti-CD45 antibody or fragment thereof is a humanized antibody.

### Targeting TGF-Beta in Fibrotic Diseases

TGF-beta (Transforming growth factor beta/TGF-β) is a type of cytokine that controls proliferation, cellular differentiation, and other functions in most cells.

TGF-beta, is a factor synthesized in a wide variety of tissues. It acts synergistically with TGF-alpha in inducing phenotypic transformation and can also act as a negative autocrine growth factor. TGF-beta has a potential role in embryonal development, cellular differentiation, hormone secretion, and immune function. TGF-beta is found mostly as homodimer forms of separate gene products TGF-β1, TGF-β2 or TGF-β3. Heterodimers composed of TGF-β1 and 12 (TGF-β1.2) or of TGF-β2 and β3 (TGF-β2.3) have been isolated. The TGF-beta proteins are synthesized as precursor proteins. The TGF-β family is part of a superfamily of proteins known as the transforming growth factor beta superfamily, which includes inhibins, activin, anti-muillerian hormone, bone morphogenetic protein, decapentaplegic and Vg-1.

There are some other names of TGF-beta/Transforming Growth Factor beta, for example, Platelet Transforming Growth Factor; Bone-Derived Transforming Growth Factor; and Milk Growth Factor.

With EndMT contributing to tissue fibrosis progression and TGF-β as pivotal mediator of the fibrotic response, an inducer of extracellular matrix deposition, and also an inducer of MR post-MI, dual intervention targeting both CD45 (expression or activity) and TGF-beta can be effective.

As used herein, the term “TGF-β inhibitor” refers to any inhibitor that blocks the interaction of TGF-β with its receptor, blocks the cell signaling elicited by the TGF-β/receptor interaction, or blocks the expression of TGF-β in the cell or release from the cell. Encomposed within “TGF-β inhibitors” are small molecule inhibitors, antagonist decoy TGF-β, synthetic chemical compound inhibitors, peptide inhibitors, peptide mimetic inhibitors, antagonist of TGF-β receptor binding, antagonist anti-TGF-β antibodies, neutralizing antibody, TGF-β and functional fragments therefrom, and nucleic acid inhibitors of TGF-β gene expression.

Some non-limiting examples of small molecule inhibitors of TGFβRs include 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), and SB-505124 (see e.g., Dacosta Byfield, S., et al., 2004; Molecular Pharmacology 65:744-52), among others.

In one embodiment, the ALK5 inhibitor 2-(3-(6-Methylpyridin-2-yl)-1H-pyrazol-4-yl)-1,5 napththyridine is used with the methods described herein. This inhibitor is also referred to herein as ALK5 inhibitor II and is available commercially from Calbiochem (Cat. No. 616452; San Diego, Calif.). In one embodiment, the inhibitor is SB 431542, an ALK-4, -5, -7 inhibitor, commercially available from Sigma (product no. 54317; Saint Louis, Mo.). SB 431542 is also referred to by the following chemical names: 4-[4-(1,3-Benzodioxol-5-yl)-5-(2-pyridinyl)-1H-imidazol-2-yl]-benzamide, 4-[4-(3,4-methylenedioxyphenyl)-5-(2-pyridyl)-1H-imidazol-2-yl]-benzamide, or 4-(5-benzol[1,3]dioxol-5-yl-4-pyridin-2-yl-1H-imidazol-2-yl)-benzamide hydrate.

Small molecules inhibitors of TGF-β signaling can be classified based on the basic scaffold of the molecule. For example, TGF-β signaling inhibitors can be based on the dihydropyrrlipyrazole-based scaffold, imidazole-based scaffold, pyrazolopyridine-based scaffold, pyrazole-based scaffold, imidazopyridine-based scaffold, triazole-based scaffold, pyridopyrimidine-based scaffold, pyrrolopyrazole-based scaffold, isothiazole-based scaffold and oxazole-based scaffold.

Inhibitors of TGF-β signaling are described in Callahan, J. F. et al., J. Med. Chem. 45, 999-1001 (2002); Sawyer, J. S. et al., J. Med. Chem. 46, 3953-3956 (20031; Gellibert, F. et al., J. Med. Chem. 47, 4494-4506 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Dumont, N, et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030,125; U.S. Pat. No. 6,943,191; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0138188 and U.S. Publication No. 2009/0036382, the contents of each which are herein incorporated by reference in their entirety.

Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, each of which are herein incorporated by reference in its entirety. Other antisense nucleic acids and siRNAs can be obtained by methods known to one of ordinary skill in the art.

Several clinical trials on fibrosis with anti-TGF-beta agents have been done or are currently running. For example in skin fibrosis a clinical trial is testing the effect of the synthetic peptide P144 (ClinicalTrials.gov Identifier: NCT00781053). P144 encodes a part of the ligand (TGF-beta) binding domain of beta-glycan and can block TGF-beta activity. In animal models P144 inhibited carbon tetrachloride induced liver fibrosis and more importantly it inhibited bleomycin induced skin fibrosis. P144, P17 and other peptide inhibitors of TGF-β are fully described in the US patent application publication No.: US20120315256 and U.S. Pat. No. 7,582,609, and the contents of each publication is incorporated herein by reference in its entirety.

Exemplary inhibitors of TGF-β signaling include, but are not limited to, AP-12009 (TGF-β Receptor type II antisense oligonucleotide), Lerdelimumab (CAT 152, antibody against TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad6, Smad7), and Cystatin C.

Inhibitors of TGF-β signaling also include molecules which inhibit TGF-β Receptor type I. Inhibitors of TGF-β Receptor type I are described in Byfield, S. D., and Roberts, A. B., Trends Cell Biol. 14, 107-111 (2004); Sawyer J. S. et al., Bioorg. Med. Chem. Lett. 14, 3581-3584 (2004); Sawyer, J. S. et al., J. Med. Chem. 46, 3953-3956 (2003); Byfield, S. D. et al., Mol. Pharmacol. 65, 744-752 (2004); Gellibert, F. et al., J. Med. Chem. 47, 4494-4506 (2004); Yingling, J. M. et al., Nature Rev. Drug Disc. 3, 1011-1022 (2004); Dumont, N, et al., Cancer Cell 3, 531-536 (2003); Tojo, M. et al., Cancer Sci. 96: 791-800 (2005); the International Pat. Publication No.: WO Publication No. 2004/026871; WO Publication No. 2004/021989; WO Publication No. 2004/026307; WO Publication No. 2000/012497; U.S. Pat. No. 5,731,424; U.S. Pat. No. 5,731,144; U.S. Pat. No. 7,151,169; U.S. Publication No. 2004/00038856 and U.S. Publication No. 2005/0245508, contents of all of which are herein incorporated in their entireties.

Exemplary inhibitors of TGF-β Receptor type I include, but are not limited to, soluble TGF-β Receptor type I; AP-11014 (TGF-β Receptor type I antisense oligonucleotide); Metelimumab (CAT 152, TGF-β Receptor type I antibody); LY550410; LY580276 (3-(4-fluorophenyl)-5,6-dihydro-2-(6-methylpyridin-2-yl)-4H-pyrrolo[1,2-b]pyrazole); LY364947 (4-[3-(2-Pyridinyl)-1H-pyrazol-4-yl]-quinoline); LY2109761; LY573636 (N-((5-bromo-2-thienyl)sulfonyl)-2,4-dichlorobenzamide); SB-505124 (2-(5-Benzo[1,3]dioxol-5-yl-2-tert-butyl-3H-imidazol-4-yl)-6-methylpyridine); SD-208 (2-(5-Chloro-2-fluorophenyl)-4-[(4-pyridyl)amino]pteridine); SD-093; KI2689; SM16; FKBP12 protein; and 3-(4-(2-(6-methylpyridin-2-yl)H-imidazo[1,2-a]pyridin-3-yl)quinolin-7-yloxy)-N,N-dimethylpropan-1-amine.

A non-limiting example of a chemical compound antagonist of TGF-beta is pirfenidone. Pirfenidone [5-methyl-1-phenyl-2(1H)-pyridone] is an orally active small molecule which is known for its anti-fibrotic action. Pirfenidone inhibits TGF-β at multiple steps: it reduces TGF-β promoter activity, TGF-β protein secretion, TGF-β-induced Smad2 phosphorylation and generation of reactive oxygen species.

CAT-152 (lerdelimumab) is a fully human TGF-beta2 neutralizing antibody with high affinity for TGF-beta2 and lower affinity for TGF-beta3. In rabbits it was capable of inhibiting scarring after glaucoma surgery. In the first clinical trials, CAT-152 showed possible effects in reducing scar formation in intractable glaucoma patients that received a trabeculectomy.

Another example is fresolimumab, a human monoclonal antibody against that inactivates all forms of transforming growth factor-β (TGF-β). Other non-limiting examples of TGF-β1 neutralizing antibody are (CAT-192, metelimumab), pan-TGF-β neutralizing antibody (GC-1008); SM16, a novel small molecule ALK5 kinase inhibitor; SB-431542; and the four major classes of TGF-β inhibitors include ligand traps (e.g. 1D11 or Fresolimumab), antisense oligonucleotides (ASO) like Trabedersen, small molecule receptor kinase inhibitors such as LY2109761 or LY2157299, and peptide aptamers (e.g. Trx-SARA). Other non-limiting examples of humanized anti-TGF-beta antibodies are found in U.S. Pat. Nos. 5,616,561, 5,783,185, 5,571,714, 7,527,791, the International Pat. Publication No.: WO2005/097832 and WO2006/086469, the contents of each are incorporated herein by reference in their entirety.

GC2008 or Fresolimumab which is the humanized version of the pan-TGF-β neutralizing antibody 1D11 is also tested in clinical trials. These trials also treat patients with diffuse systemic sclerosis (ClinicalTrials.gov Identifier: NCT01284322).

In a phase I/II trial CAT-192, a TGF-β1 neutralizing antibody did not show effect on early stage diffuse cutaneous systemic sclerosis, together with more adverse events occurred in the CAT-192 treated groups (ClinicalTrials.gov Identifier: NCT00043706). Nevertheless this antibody is now being tested in patients with myelofibrosis (ClinicalTrials.gov Identifier: NCT01291784).

### Compositions, Formulation and Administration

In one embodiment, provided herein is an inhibitor of CD45 for use in the inhibition of pathological EndMT in an endothelial cell. In another embodiment, provided herein is an inhibitor of CD45 for use in the manufacture of medicament for the inhibition of pathological EndMT in an endothelial cell.

In one embodiment, provided herein is an inhibitor of CD45 for use in the inhibition of pathological EndMT in a mammal. In another embodiment, provided herein is an inhibitor of CD45 for use in the manufacture of medicament for the inhibition of pathological EndMT in a mammal.

In one embodiment, provided herein is an inhibitor of CD45 for use in the prevention, treatment or management of a medical condition that involved pathological EndMT.

In one embodiment, provided herein is an inhibitor of CD45 for use in the manufacture of medicament for the prevention, treatment or management of a medical condition that involved pathological EndMT.

In one embodiment, the CD45 inhibitor is used in conjunction with a TGF-β inhibitor.

In another embodiment, the CD45 inhibitor is used in conjunction with an anti-fibrosis agent, e.g., human recombinant decorin.

In one embodiment, provided herein is a composition comprising a CD45 inhibitor for use in the inhibition of EndMT in an endothelial cell.

In one embodiment, provided herein is a composition comprising a CD45 inhibitor for use in the inhibition of EndMT in a mammal.

In one embodiment of a composition described, the composition further comprises an inhibitor of TGF-beta.

In one embodiment of a composition described, the composition further comprises an inhibitor of fibrosis (i.e., an anti-fibrosis agent or anti-fibrotic agent) wherein the inhibitor of fibrosis is not an inhibitor of TGF-beta or a CD45 inhibitor.

In one embodiment, provided herein is a composition comprising an inhibitor of CD45 and an inhibitor of TGF-beta.

In one embodiment, provided herein is a composition comprising an inhibitor of CD45, an inhibitor of TGF-beta, and an inhibitor of fibrosis wherein the inhibitor of fibrosis is not an inhibitor of TGF-beta or a CD45 inhibitor.

In one embodiment, provided herein is a composition comprising an inhibitor of CD45 and an inhibitor of fibrosis wherein the inhibitor of fibrosis is not an inhibitor of TGF-beta or a CD45 inhibitor.

In one embodiment, provided herein is a composition comprising an inhibitor of TGF-beta and an inhibitor of fibrosis wherein the inhibitor of fibrosis is not an inhibitor of TGF-beta or a CD45 inhibitor.

Such compositions are used in the inhibition of EndMT in an endothelial cell. Such compositions can also be used in the inhibition of EndMT in a mammal.

In one embodiment, provided herein is a composition comprising an inhibitor of CD45 and an inhibitor of TGF-beta for use in the inhibition of EndMT in an endothelial cell or in the inhibition of EndMT in a mammal.

In one embodiment, provided herein is a composition comprising an inhibitor of CD45, an inhibitor of TGF-beta, and an inhibitor of fibrosis wherein the inhibitor of fibrosis is not an inhibitor of TGF-beta or a CD45 inhibitor, and wherein the composition is for use in the inhibition of EndMT in an endothelial cell or in the inhibition of EndMT in a mammal.

In one embodiment of a composition described, the composition further comprises a pharmaceutically acceptable carrier.

In one embodiment of a composition described, the composition further comprises an inhibitor of tissue fibrosis wherein the inhibitor of fibrosis is not an inhibitor of TGF-beta or a CD45 inhibitor.

By “fibrosis” the term includes any condition characterized by the formation or development of excess fibrous connective tissue, excess extracellular matrix, excess scarring or excess collagen deposition in an organ or tissue as a reparative or reactive process. Fibrosis related diseases include: idiopathic pulmonary fibrosis; skin fibrosis, such as scleroderma, post-traumatic and operative cutaneous scarring; eye fibrosis, such as sclerosis of the eyes, conjunctival and corneal scarring, pterygium; cystic fibrosis of the pancreas and lungs; endomyocardial fibrosis; idiopathic myocardiopathy; cirrhosis; mediastinal fibrosis; progressive massive fibrosis; proliferative fibrosis; neoplastic fibrosis. Tuberculosis may cause fibrosis of the lungs. Therefore fibrosis occurs in a wide range of organs and tissues, including the lung, eye, skin, kidney, liver, pancreas and joints.

In one embodiment, provided herein is a pharmaceutical composition comprising an inhibitor of CD45 and a pharmaceutically acceptable carrier for use in the inhibition of EndMT in an endothelial cell.

In one embodiment, provided herein is a pharmaceutical composition comprising an inhibitor of CD45 and a pharmaceutically acceptable carrier for use in the inhibition of EndMT in a mammal.

In one embodiment, provided herein is a pharmaceutical composition comprising an inhibitor of CD45, an inhibitor of TGF-beta, and a pharmaceutically acceptable carrier for use in the inhibition of EndMT in an endothelial cell.

In one embodiment, provided herein is a pharmaceutical composition comprising an inhibitor of CD45, an inhibitor of TGF-beta, and a pharmaceutically acceptable carrier for use in the inhibition of EndMT in a mammal.

In one embodiment of a pharmaceutical composition described, the pharmaceutical composition further comprises an inhibitor of tissue fibrosis or an anti-fibrosis or anti-fibrotic agent, wherein the inhibitor of fibrosis is not an inhibitor of TGF-beta or a CD45 inhibitor. Example of an inhibitor of fibrosis that is not an inhibitor of TGF-beta or a CD45 inhibitor is the human recombinant decorin described in Fukushima K. et al, Am J Sports Med. 2001, 29(4):394-402.

Numerous anti-fibrosis agents are known in the art. For examples, U.S. Pat. Nos. 4,997,854, 5,891,477, 5,993,845, 6,750,028, 7,026,287, 7,572,440, 7,132,098, 8,404,268, 8,624,056, 9,062,076, U.S. Publication Nos.: 2005/0175703, 2005/0178395, 2005/0178396, 2005/0182463, 2005/0183731, 2005/0186244, 2005/0187140, 2005/0196421, 2005/0208095, 2008/0159995, 2010/0003237, 2012/0238502, and 2012/0052040, and the contents of each are incorporated herein by reference in their entirety.

Non-limiting anti-fibrotic agents include pancreatic elastase, elastase-2a, elastase-2b, neutrophil elastase, proteinase-3, endogenous vascular elastase, cathepsin G, mast cell chymase, mast cell tryptase, plasmin, thrombin, granzyme B, cathepsin S, cathepsin K, cathepsin L, cathepsin B, cathespin C, cathepsin H, cathespin F, cathepsin G, cathepsin 0, cathepsin R, cathepsin V (cathepsin 12), cathepsin W, calpin 1, calpin 2, chondroitinase ABC, chondroitinase AC, hyaluronidase, chymopapain, chymotrypsin, legumain, cathepsin Z (cathepsin X), cathepsin D, cathepsin E, collagenase, matrix metalloproteinases, such as for example, MMP-1 (collagenase-1), MMP-9, MMP-7 (matrilysin), MMP-8 (collagenase-2), MMP-13 (collagenase-3), MMP-18 (collagenase-4), MMP-2 (gelatinase a), MMP-9 (gelatinase b), MMP-3 (stromelysin-1), MMP-10 (stromelysin-2), MMP-11 (stromelysin-3), MMP-7 (matrilysin), MMP-26 (matrilysin), MMP-12 (metalloelastase), MMP-14 (MT1-MMP), MMP-15 (MT2-MMP), MMP-16 (MT3-MMP), MMP-17 (MT4-MMP), MMP-24 (MT5-MMP) transmembrane, MMP-25 (MT6-MMP), gpl anchor, MMP-19, MMP-20 (enamelysin), MMP-x, MMP-23, MMP-27, MMP-28 (epilysin), ADAMTS-1, ADAMTS-2, ADAMTS-3, ADAMTS-4 (aggrecanase-1), ADAMTS-5(aggrecanase-2), ADAMTS-14, papain, subtilisin, subtilisin A, heparanase. tyrosine kinase inhibitors: imatinib mesylate, dasantinib, nilotinib, inhibitors of PKC-delta and other kinases, HMG-CoA inhibitors, angiotensin inhibitors: angiotensin-converting enzyme inhibitors, angiotensin-II receptor antagonist, pirfenidone, rosiglitazone, cannabinoid receptor, trabedersen, lerdelimumab, metelimumab, mycophenolate mofetil, interferon, or a combination thereof. In some embodiments, the antifibrotic factors include, but are not limited to, interleukins, interferons, cytokines, chemokines, chemotactic molecules, macrophages, lymphocytes, tumor necrosis factor alpha (TNF-alpha), T cells, interferon gamma (IFN-gamma), relaxin, hormones (e.g., progesterone, estrogen, testosterone, growth hormone, thyroid hormone, parathyroid hormone, etc.) or a combination thereof.

In some embodiments, anti-fibrotic agents also include anti-inflammatory agents such as a statin, sulindac, sulfasalazine, naroxyn, diclofenac, indomethacin, ibuprofen (e.g., NSIDS), flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone (e.g., steroids), prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. Specifically, it refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

As used herein, the terms “administering,” refers to the placement of an inhibitor of CD45 and/or TGF-beta, or compositions described into a mammal by a method or route which results in at least partial localization of the said inhibitor at a desired site, the endothelial cells. In particular, endothelial cells that are undergoing EndMT. The inhibitor of CD45 and/or TGF-beta can be administered by any appropriate route which results in an effective treatment in the subject.

As used herein, the term “comprising” or “comprises” is used in reference to methods, and respective component(s) thereof, that are essential to the invention, yet open to the inclusion of unspecified elements, whether essential or not. The use of “comprising” indicates inclusion rather than limitation.

Therapeutic compositions or pharmaceutical compositions can be formulated for passage through the blood-brain barrier or direct contact with the endothelium. In some embodiments, the compositions can be formulated for systemic delivery. In some embodiments, the compositions can be formulated for delivery to specific organs, for example but not limited to the liver, spleen, the bone marrow, the mitral valve in the heart and the skin. Therapeutic compositions or pharmaceutical compositions can be formulated for aerosol application by inhalation the lung. Alternatively, the therapeutic compositions or pharmaceutical compositions can also be formulated for a transdermal delivery, e.g. a skin patch. Therapeutic compositions or pharmaceutical compositions can be enteric coated and formulated for oral delivery. Therapeutic compositions or pharmaceutical compositions can be encapsulated in liposomes or nanoparticles and formulated for slow sustained delivery in vivo. Alternatively, the therapeutic compositions or pharmaceutical compositions is be formulated for targeted delivery, eg., encapsulated in liposomes or nanoparticles that are designed and feature targeting moiety to on the liposomes or nanoparticles. For example, targeted endothelial cells by way of CD31 or other known endothelial markers such as adhesion molecules. In other embodiments, the nanoparticles comprises charged polymers containing aromatic sulfonate have pronounced affinity for caveolae, which are highly expressed by endothelial cells (Julia Voigt et al., PNAS, 2013, vol. 111 no. 8, pp 2942-2947).

The inhibitor of CD45, the inhibitor of TGF-beta, and the compositions described herein can be administered by any known route. By way of example, the inhibitor of CD45, the inhibitor of TGF-beta, and the compositions described herein can be administered by a mucosal, pulmonary, topical, or other localized or systemic route (e.g., enteral and parenteral). The inhibitor of CD45 and/or the inhibitor of TGF-beta, may be administered by any convenient route, for example by infusion or bolus injection, by absorption through epithelial or mucocutaneous linings (e.g., oral mucosa, rectal and intestinal mucosa, etc.) and may be administered together with other biologically active agents.

Routes of administration include, but are not limited to aerosol, direct injection, intradermal, transdermal (e.g., in slow release polymers), intravitreal, intramuscular, intraperitoneal, intravenous, subcutaneous, intranasal, epidural, topical, oral, transmucosal, buccal, rectal, vaginal, transdermal, intranasal and parenteral routes. “Parenteral” refers to a route of administration that is generally associated with injection, including but not limited to intraorbital, infusion, intraarterial, intracapsular, intracardiac, intradermal, intrahepatic, intrarogan, intramuscular, intraperitoneal, intrapulmonary, intraspinal, intrasternal, intrathecal, intrauterine, intravenous, subarachnoid, subcapsular, subcutaneous, transmucosal, or transtracheal. Any other therapeutically efficacious route of administration can be used, for example, infusion or bolus injection, absorption through epithelial or mucocutaneous linings, or by gene therapy wherein a DNA molecule encoding the therapeutic protein or peptide is administered to the patient, e.g., via a vector, which causes the protein or peptide to be expressed and secreted at therapeutic levels in vivo. In various embodiments, administration can be inhaled in to the lung via aerosol administration, e.g. with nebulization. Administration also can be systemic or local. Intratumoral delivery is also included.

For example, the inhibitor of CD45 and/or the inhibitor of TGF-beta, can be administered as a formulation adapted for passage through the blood-brain barrier or direct contact with the endothelium. In some embodiments, the inhibitor of CD45, the inhibitor of TGF-beta, or composition described can be administered as a formulation adapted for systemic delivery. In some embodiments, the inhibitor of CD45, the inhibitor of TGF-beta, or composition described can be administered as a formulation adapted for delivery to specific organs, for example but not limited to the liver, spleen, the bone marrow, and the skin.

In addition, the inhibitor of CD45 and/or the inhibitor of TGF-beta, or compositions described herein can be administered together with other components of biologically active agents, such as pharmaceutically acceptable surfactants (e.g., glycerides), excipients (e.g., lactose), carriers, diluents and vehicles.

In another embodiment of any methods or compositions described, the inhibitor of CD45, the inhibitor of TGF-beta described herein in administered together with other therapeutics for the various medical conditions when the inhibitor of CD45 and/or the inhibitor of TGF-beta is/are administered for preventing, treatment and/or management of a medical condition involving pathological EndMT.

The inhibitor of CD45 and/or the inhibitor of TGF-beta or compositions described herein can be administered therapeutically to a subject prior to, simultaneously with (in the same or different compositions) or sequentially with the administration of at least one other cancer therapy or one other therapeutics for the various medical conditions involving pathological EndMT. For example, the addition cancer therapy is radiation or chemotherapy or proton therapy. The inhibitor of CD45 and/or the inhibitor of TGF-beta or compositions described herein antagonists can be administered as adjunctive and/or concomitant therapy to a cancer therapy.

For parenteral (e.g., intravenous, subcutaneous, intramuscular) administration, inhibitor of CD45, the inhibitor of TGF-beta, or compositions described herein can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and non-aqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques.

The dosage administered to a subject will vary depending upon a variety of factors, including the pharmacodynamic characteristics of the particular antagonists, and its mode and route of administration; size, age, sex, health, body weight and diet of the recipient; nature and extent of symptoms of the disease being treated, kind of concurrent treatment, frequency of treatment, and the effect desired.

Usually a daily dosage of active ingredient can be about 0.01 to 500 milligrams per kilogram of body weight. Ordinarily 1 to 40 milligrams per kilogram per day given in divided doses 1 to 6 times a day or in sustained release form is effective to obtain desired results. The active ingredient will ordinarily be present in an amount of about 0.5-95% by weight based on the total weight of the composition. Second or subsequent administrations can be administered at a dosage which is the same, less than or greater than the initial or previous dose administered to the individual.

A second or subsequent administration is preferably during or immediately prior to relapse or a flare-up of the disease or symptoms of the disease. For example, second and subsequent administrations can be given between about one day to 30 weeks from the previous administration. Two, three, four or more total administrations can be delivered to the individual, as needed.

The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

Efficacy testing can be performed during the course of treatment using the methods described herein. Measurements of the degree of severity of a number of symptoms associated with a particular ailment are noted prior to the start of a treatment and then at later specific time period after the start of the treatment.

The precise dose to be employed in the formulation of the agent will also depend on the route of administration, and the seriousness of the disease or disorder, and should be decided according to the judgment of the practitioner and each patient's circumstances. Effective doses may be extrapolated from dose-response curves derived from in vitro or animal model test systems.

Efficacy testing can be performed during the course of treatment using the methods described herein. Measurements of the degree of severity of a number of symptoms associated with a particular ailment are noted prior to the start of a treatment and then at later specific time period after the start of the treatment. For example, when treating an autoimmune disease such as rheumatoid arthritis, the severity of joint pain can be scored from a number of 1-10, with a score of 1 representing mild discomfort and a score of 10 represent constant unbearable pain with or without movement; the range of motion of an affected joint can also are be measured as a degree of angle for which that joint can move. The joint pain and range of motion are noted before and after a treatment. The severity of joint pain and range of motion after the treatment are compared to those before the treatment. A decrease in the pain score and/or an increase in the degree of angle of joint movement indicate that the treatment is effective in reducing inflammation in the affected joint, thereby decreasing pain and improving joint movement.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective dose can include a single treatment or a series of treatments. Estimates of effective dosages and in vivo half-lives for the inhibitors used can be made using conventional methodologies or on the basis of in vivo testing using an appropriate animal model, as known in the art, or as described herein. Preferred dosages for the inhibitors used are readily determinable by those of skill in the art by a variety of means.

The present invention can be defined in any of the following numbered paragraphs:
[1] An inhibitor of CD45 for use in the inhibition of pathological endothelial-to-mesenchymal transition (EndMT) in an endothelial cell.
[2] An inhibitor of CD45 for use in the manufacture of medicament for the inhibition of pathological endothelial-to-mesenchymal transition (EndMT) in an endothelial cell.
[3] An inhibitor of CD45 for use in the inhibition of pathological EndMT in a mammal.
[4] An inhibitor of CD45 for use in the manufacture of medicament for the inhibition of pathological EndMT in a mammal.
[5] An inhibitor of CD45 for use in the prevention, treatment or management of a medical condition that involved pathological EndMT.
[6] An inhibitor of CD45 for use in the manufacture of medicament for the prevention, treatment or management of a medical condition that involved pathological EndMT.
[7] The CD45 inhibitor of any one of the preceding paragraphs where the CD45 inhibitor is used in conjunction with a TGF-β inhibitor.
[8] The CD45 inhibitor of any one of the preceding paragraphs where the CD45 inhibitor is used in conjunction with an anti-fibrosis agent.
[9] A composition comprising a CD45 inhibitor of any one of the preceding paragraphs for use in the inhibition of EndMT in an endothelial cell or for use in the inhibition of EndMT in a mammal.

[10] A composition comprising an inhibitor of CD45 and an inhibitor of TGF-beta.

[11] The composition of any one of the preceding composition paragraphs, wherein the composition further comprises a pharmaceutically acceptable carrier.

[12] The composition of any one of the preceding composition paragraphs, wherein the composition further comprises an inhibitor of tissue fibrosis.

[13] A method of inhibiting pathological endothelial-to-mesenchymal transition (EndMT) comprising contacting an endothelial cell with an inhibitor of protein tyrosine phosphatase, receptor type, C, (PTPRC or CD45) of paragraphs 1-8, or a composition of paragraphs 9-12 comprising a CD45 inhibitor.

[14] A method of inhibiting a pathological endothelial-to-mesenchymal transition (EndMT) in a mammal comprising administering an effective amount of an inhibitor of protein tyrosine phosphatase, receptor type, C, (PTPRC or CD45) of paragraphs 1-8, or a composition of paragraphs 9-12 comprising a CD45 inhibitor to the mammal.

[15] The method of paragraphs 13 or 14, the CD45 inhibitor of paragraphs 1-8, and the compositions of paragraphs 9-12, wherein the pathological EndMT occurs in a disease or injury.

[16] The method of paragraphs 13, 14, or 15, the CD45 inhibitor of paragraphs 1-8, and the compositions of paragraphs 9-12, wherein the pathological EndMT produces excessive or undesirable tissue remodeling.

[17] The method of any one of paragraphs 13-16, the CD45 inhibitor of paragraphs 1-8, and the compositions of paragraphs 9-12, wherein the pathological EndMT produces excessive or undesirable fibrosis.

[18] The method of any one of paragraphs 13-17, the CD45 inhibitor of paragraphs 1-8, and the compositions of paragraphs 9-12, wherein the pathological EndMT occurs in conditions selected from the group consisting of mitral regurgitation (MR), myocardial infarction (MI), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, hepatic fibrosis, cirhhosis, fibrosis induced transplant antibody mediated rejection, cerebral cavernous malformations, arteriosclerosis, intimal hyperplasia in vein-to-arterial graft and pulmonary fibrosis.
[19] The method of any one of paragraphs 13-18, the method further comprising detecting the expression of CD45 in the endothelial cells.
[20] The method of any one of paragraphs 13-19, the method further comprising detecting the presence of EndMT.
[21] The method of paragraph 20, wherein the detection of EndMT comprises assessing expression of at least one biomarker selected from the group consisting of Fascin1, Vimentin and Hsp47.
[22] The method of any one of paragraphs 13-21, wherein the CD45 inhibitor is targeted to an endothelial cell, wherein the endothelial cell is undergoing EndMT.
[23] The method of any one of paragraphs 13-22, wherein the CD45 inhibitor is targeted to endothelial cells expressing CD45.
[24] The method of any one of paragraphs 13-23, the method further comprising contacting the endothelial cell or administering to the mammal a TGFβ inhibitor.
[25] The method of any one of paragraphs 13-24, the method further comprising contacting the endothelial cell or administering to the mammal an anti-fibrosis agent wherein the anti-fibrosis agent is not a CD45 inhibitor or a TGFβ inhibitor.

This invention is further illustrated by the following example which should not be construed as limiting. The contents of all references cited throughout this application, as well as the figures and tables are incorporated herein by reference.

Those skilled in the art will recognize, or be able to ascertain using not more than routine experimentation, many equivalents to the specific embodiments of the invention described herein. Such equivalents are intended to be encompassed by the following claims.

### Example

### Materials and Methods

Animal Model—

A total of nine Dorsett hybrid sheep were analyzed. The IMI sheep (n=5) had ligation of their second and third obtuse marginal branches of the left circumflex coronary artery. Two- and 3-dimensional echocardiography was performed before MI and 30-60 minutes following MI. Six months post-MI, echocardiograms were repeated to assess MR and the animals euthanized and their MVs excised. The excised MV leaflet tissue was immediately submerged in a solution of 5% heat-inactivated fetal bovine serum (FBS), 4% Penicillin/Streptomycin/Amphotericin B, 1% L-Glutamine and 0.2% gentamycin sulfate in EBM-2 medium (Lonza Inc., GA, USA, #CC-3156), and kept on ice at 4° C. until processed. MV from five (n=5) age and weight-matched sheep who underwent sham procedures (thoracotomy without infarction) were analyzed as controls. Echocardiographic measurements are shown in Table 3. Three-dimensional (3D) echocardiographic analysis included LV end-systolic and end-diastolic volumes integrated from multiple rotated views derived from the full 3D dataset using Omni4D software; infarct size as endocardial surface area (ESA) measured at end diastole based on visualized wall motion hinge points; and total LV remodeling reflected by the increase in total LV ESA from immediately to 6 months post-MI³⁷⁻⁴⁰. In addition, mitral valves from normal sheep (n=3) were analyzed. Animals were monitored by qualified AAALAC-certified veterinary staff. These studies conform to National Institutions of Heath guidelines for animal care and received Institutional Animal Care Committee approval.

Immunohistochemistry—

Excised MVs were frozen in OCT compound (Sakura Finetek, Tokyo, Japan), sectioned and stained with primary mouse anti-sheep CD45 antibody (AbD Serotec, NC, USA, cat# MCA2220GA) by the avidin-biotin-peroxidase method or fluorescence double-labeling for von Willebrand Factor (vWF; DAKO, Calif., USA, cat#, A0082) and CD45 (AbD Serotec, NC, USA) as described¹⁰.

Flow Cytometry—

Ovine MVs were minced and digested for 30 minutes at 37° C. with Liberase (Roche Diagnostics, IN, USA, #5401119001), a blend of purified collagenases I and II. Isolated cells were fixed using Flow Cytometry Fixation Buffer (R&D Systems, MN, USA, #FC004) and labeled in Flow Cytometry Permeabilization/Wash Buffer I (R&D Systems, MN, USA, #FC005) for 45 minutes (100,000 cells/100 μl Buffer I). Murine anti-human CD45-FITC or -APC (1:50; AbD Serotec, NC, USA, # MCA2220F and #559864), VE-cadherin-PE or -FITC (1:100; R&D Systems, MN, USA, # FAB9381P and AbD Serotec, NC, USA, # AHP628F), and α-smooth muscle actin (αSMA)-APC or -PE (1:100; R&D Systems, MN, USA, # IC1420P and # IC1420A) were used. All antibodies were shown to cross-react with their ovine homologs.

Cell Culture—

Ovine mitral VEC clones and carotid artery endothelial cells (CAEC) were isolated 11 and grown on 1% gelatin-coated dishes in EBM-2 medium (Lonza Inc., GA, USA #CC-3156) supplemented with 10% heat-inactivated FBS, 1% glutamine-penicillin-streptomycin sulfate (Life Technologies, cat#10378-016) and 2 ng/ml basic fibroblast growth factor (Roche LifeScience, IN, USA, cat #11123149001), henceforth referred to as EBM-B.

EndMT Assay—

Ovine mitral VEC and CAEC were plated at 10,000 cells/cm² on gelatin-coated dishes. After 24 hours, EBM-B was replaced with fresh EBM-B containing 1 ng/ml human TGFβ1 (R&D Systems, MN, USA). Cells were harvested with Liberase (100l/cm²) 96 hours later, and used for flow cytometry. Cells were analyzed simultaneously for VE-cadherin, α-SMA and CD45 by flow cytometry as described above.

Quantitative PCR (qPCR)—

Ovine mitral VEC and CAEC were subjected to the EndMT assay in the presence or absence of 0.5 μmol/L N-(9,10-dioxo-9,10-dihydro-phenanthren-2-yl)-2,2-dimethyl propionamide, a CD45-selective protein tyrosine phosphatase (PTPase) inhibitor (EMD Millipore Sigma, MA, USA#540215)²⁷. Total cellular RNA was extracted from mitral VEC clones (C5, D1, E5, E10 and E10-2) and CAEC (n=2) with an RNeasy Micro extraction kit (Qiagen, Valencia, Calif., #74004). Reverse transcriptase reactions were performed using an iScript cDNA Synthesis Kit (Bio-Rad, CA, USA #170-8890). qPCR was performed using Kapa Sybr Fast ABI Prism 2×qPCR Master Mix (KAPA BioSystems, MA, USA # KK4604). Amplification was carried out in an ABI 7500 (Applied Biosystems, Foster City, Calif.). A standard curve for each gene was generated to determine amplification efficiency. RPS9 was used as housekeeping gene expression reference. Fold increases in gene expression were calculated according to 2 delta Ct method^{28,29}, with each amplification reaction performed in triplicate.

Cellular Migration Assay—

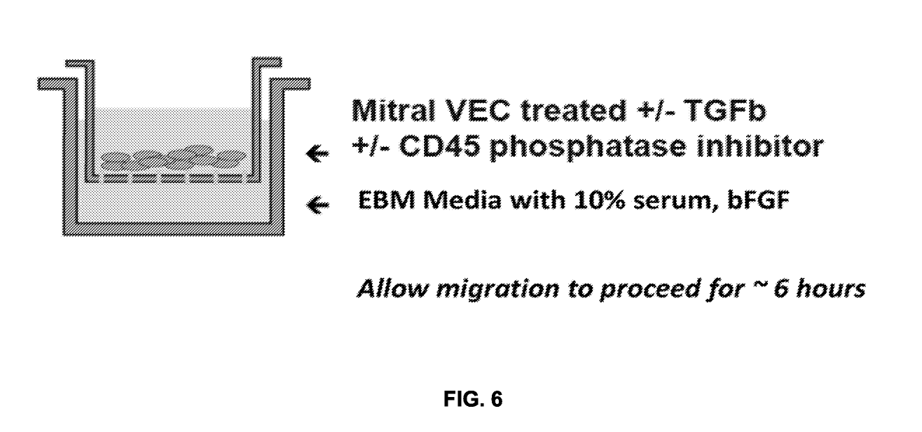

Mitral VEC clones (D1, E5, E10-2) or CAEC were treated±TGFβ for 4 days to induce EndMT. The cells were treated for 30 minutes±the CD45-selective PTPase inhibitor (1 mol/L)21 prior to trypsinization. 20,000 cells (±PTPase inhibitor 1 mol/L) in 0.1% BSA/EBM-2 (Lonza) were placed in the upper chamber of 6.5 mm Transwells containing fibronectin-coated (0.2 ug/cm2) polycarbonate membranes with 8.0 μm pores. The lower chambers contained 0.1% BSA/EBM-2 media alone or EBM with serum and basic FGF as a chemoattractants. Cells were allowed to migrate for 6 hours at 37° C. (FIG. 6) Cells that migrated through the pores were fixed with methanol and stained with Eosin-Y, Azure A and Methylene Blue for visualization and quantification using Three Step Stain Set (VWR, PA, USA #48218-567). In parallel, an aliquot of cells used for the migration assay were also analyzed for CD45 by flow cytometry to verify response to TGFβ1.

Statistics—

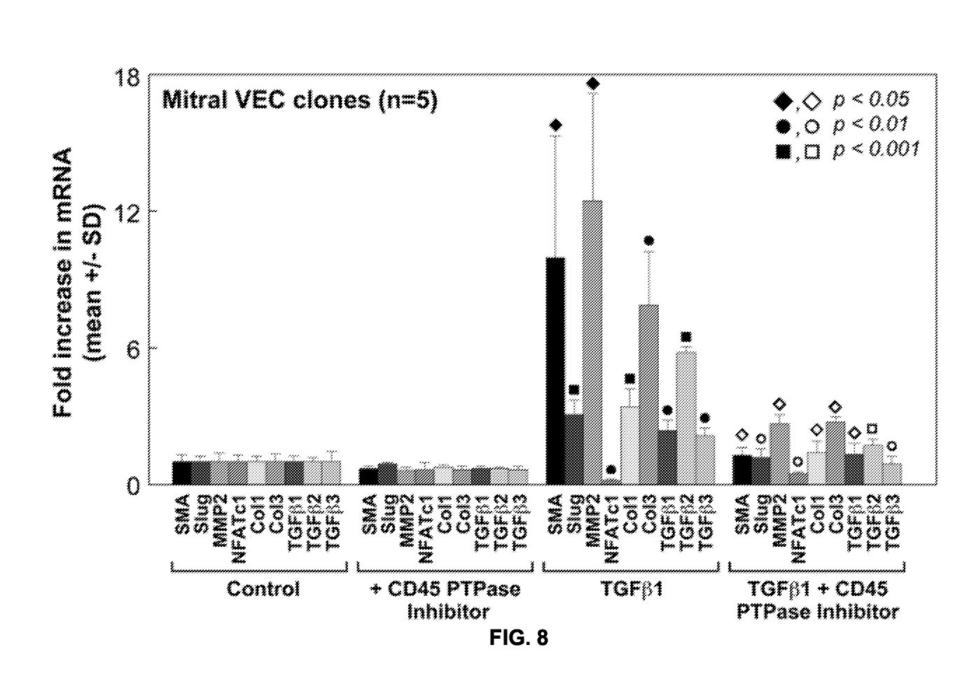

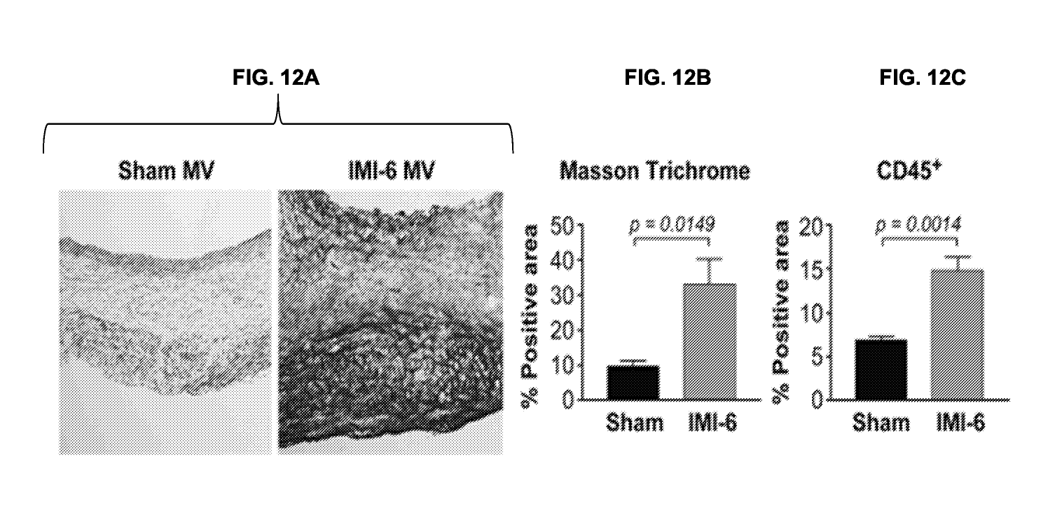

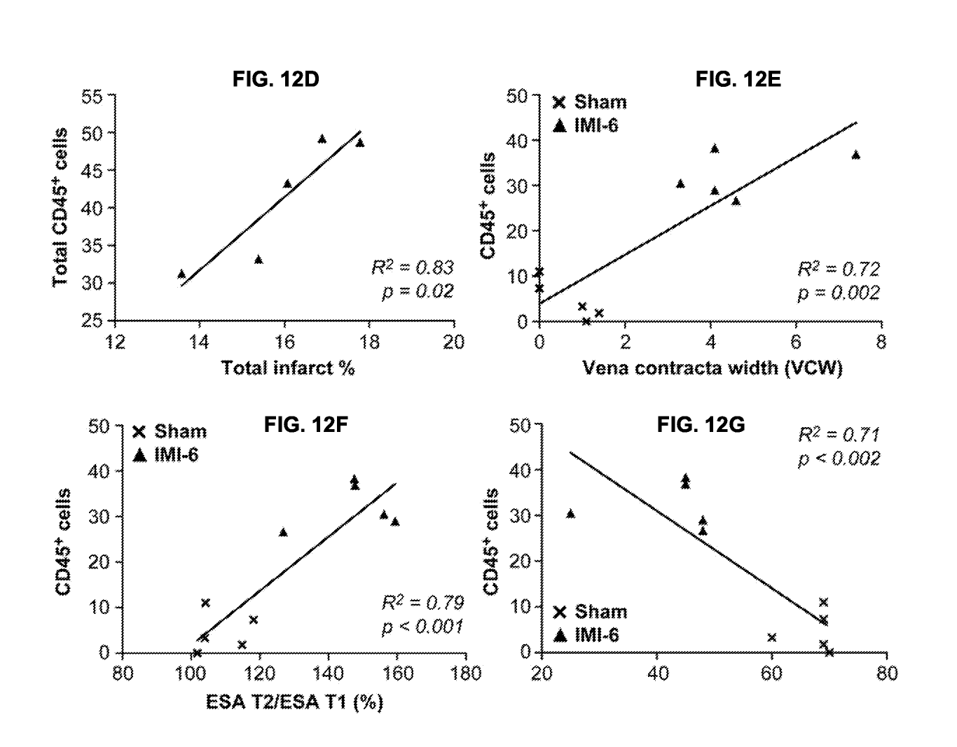

Sample variances were analyzed by Fisher tests to determine equal or non-equal variances. A Fisher p-value >0.05 was considered equal variance. Two-tailed, two sample t-tests were performed. In FIGS. 8, 9, 10A, 10B, 11A and 11B, fold changes are reported as mean±standard deviation (SD). In FIGS. 8, 9, 10A, and 10B, qPCR data from 5 different mitral valve VEC clones were standardized as described Willems³⁰. In FIGS. 8, 9, 10A, 10B, 11A and 11B, data were analyzed by one-way ANOVA, Fisher Tests, and two-tailed two independent sample T-Tests. Statistical programs were from Excel and XLStat Pro. In FIG. 12D-12G, linear regression analysis was performed and the R-squared value was calculated to see how well the regression line fit the data. P<0.05 was considered significant.

### Results

The Pan-Hematopoietic Marker CD45 is Expressed in Mitral Valve Endothelium Post-MI—

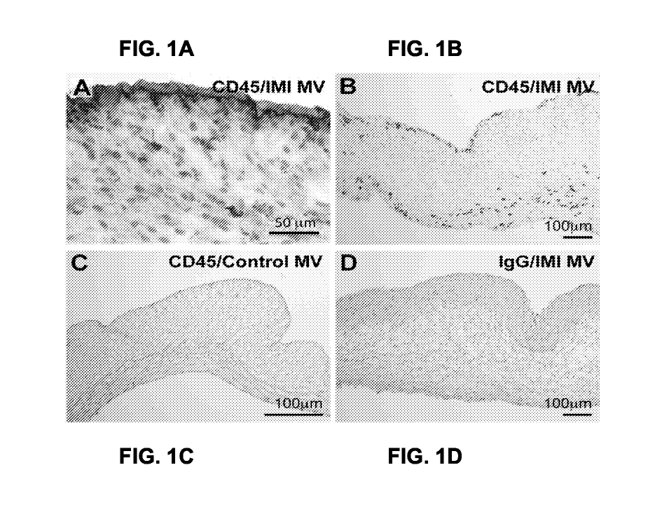

A total of 5 sheep underwent IMI and were euthanized 6 months after the procedure. All infracted sheep developed MR, as determined by color-doppler echocardiography at the time of sacrifice. MV leaflet area and thickness were increased in infarcted sheep compared to normal MV from healthy sheep (n=3) (data not shown). To assess the presence of leukocytes, MVs were stained for CD45, a receptor-like protein tyrosine phosphatase expressed in all leukocytes. Strong staining for CD45 was seen on the endothelial and sub-endothelial layers on the atrial side of the IMI MVs as well as the interstitial regions. CD45 staining was less prominent in control MVs (FIGS. 1A and 1B). Double immunostaining of IMI MVs showed CD45 co-localized with von Willebrand Factor, a glycoprotein expressed in endothelial cells¹² (data not shown). This indicated that mitral VECs might contribute to the CD45+ cell population in IMI MVs. CD45 was also partially co-localized with αSMA+ cells in IMI MV leaflets (data not shown). Based on this it is hypothesized that some of the CD45+ cells could be fibrocytes or myeloid fibroblasts, circulating myofibroblast precursors that express αSMA and produce collagen^{13,14}. αSMA and increased collagen are both hallmarks of MV adaptation to IMR⁵.

Quantification of CD45-Positive Cells in MV Endothelial, Interstitial and Hematopoietic Populations by Triple-Label Flow Cytometry—

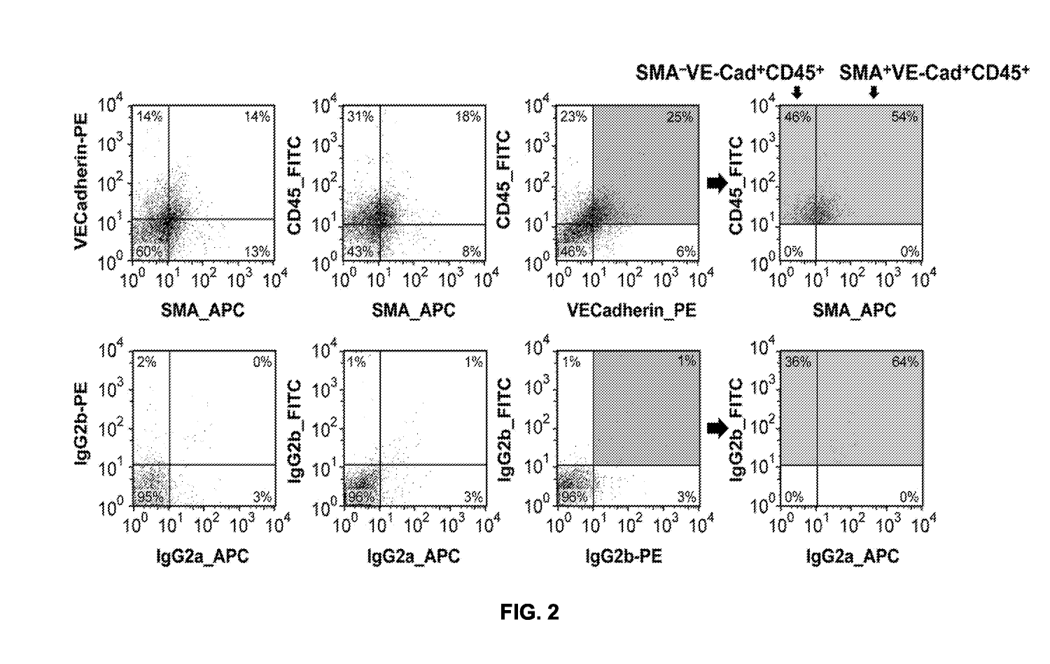

A method was devised to gently release cells from MV leaflet tissue immediately after removal from experimental models to determine the relative proportions of endothelial, interstitial and hematopoietic cell populations. Leaflet tissue was minced into small pieces and digested with mix of type I and II collagenases for 30 minutes at 37° C. to generate a single cell suspension. Cells were permeabilized, and simultaneously incubated with anti-VE-cadherin-PE (endothelial marker), anti-CD45-FITC (hematopoietic/fibrocyte marker) and anti-α-SMA-APC (myofibroblast/fibrocyte marker). This allowed us to identify and quantify 8 distinct cell populations (FIG. 2 and Table 1). The co-expression of CD45 in VE-cadherin-positive endothelial cells confirmed the CD45-positive endothelium described above. Furthermore, VE-cadherin+/CD45+/αSMA− and VE-cadherin+/CD45+/αSMA+ cells were significantly increased in IMI MVs compared to normal MVs (Table 1). There was a concomitant decrease in VE-cadherin+/CD45−/αSMA− cells in IMI MVs compared to normal MVs (Table 1).

Cells with a phenotype consistent with quiescent valve interstitial cells (VICs)—VE-cadherin−/CD45−/αSMA− were the most abundant in the MV but were not significantly different between IMI and normal MVs (Table 1). Activated VICs (VE-cadherin−/CD45−/αSMA+) were detected in both normal and IMI valves but did not differ significantly by this method of analysis. Hematopoietic cells, defined as VE-cadherin−/CD45+/αSMA−, were significantly increased in IMI MV as were fibrocytes, defined as VE-cadherin−/CD45+/αSMA+. The statistically significant increases in all MV endothelial cells (VECs) expressing CD45 and non-ECs expressing CD45, expressed as a percentage of total cells released from the leaflet tissue, (data not shown). From Table 1, one can calculate that MV ECs constituted 64% of the total CD45+ cells, hematopoietic cells 21% and fibrocytes 14% in this 6 month IMI model.

TGFβ1 Induces CD45 in Concert with EndMT in Mitral VEC Clones—

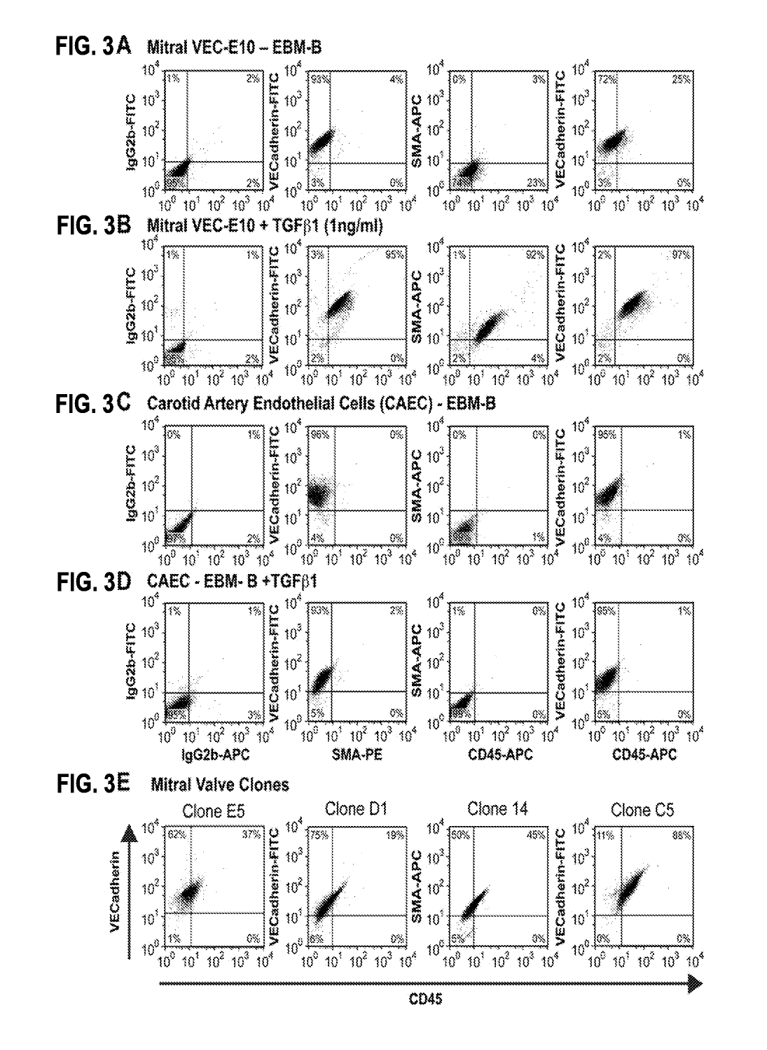

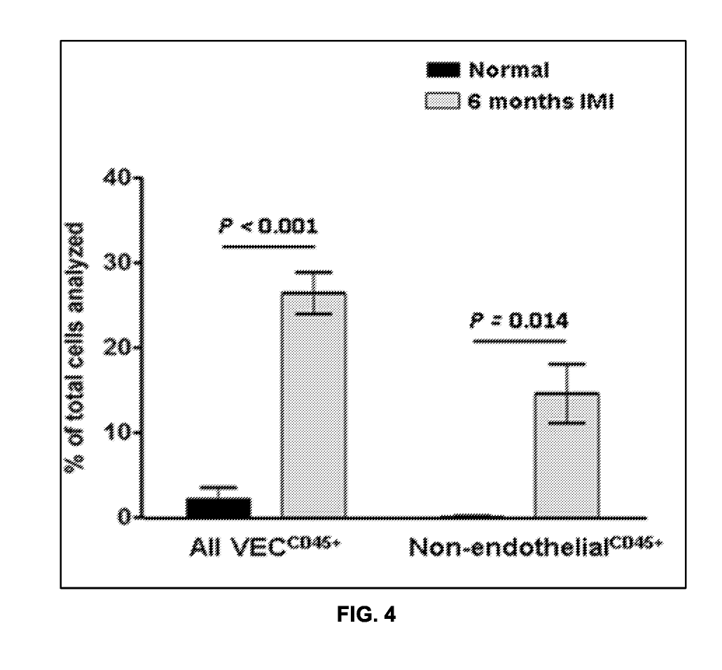

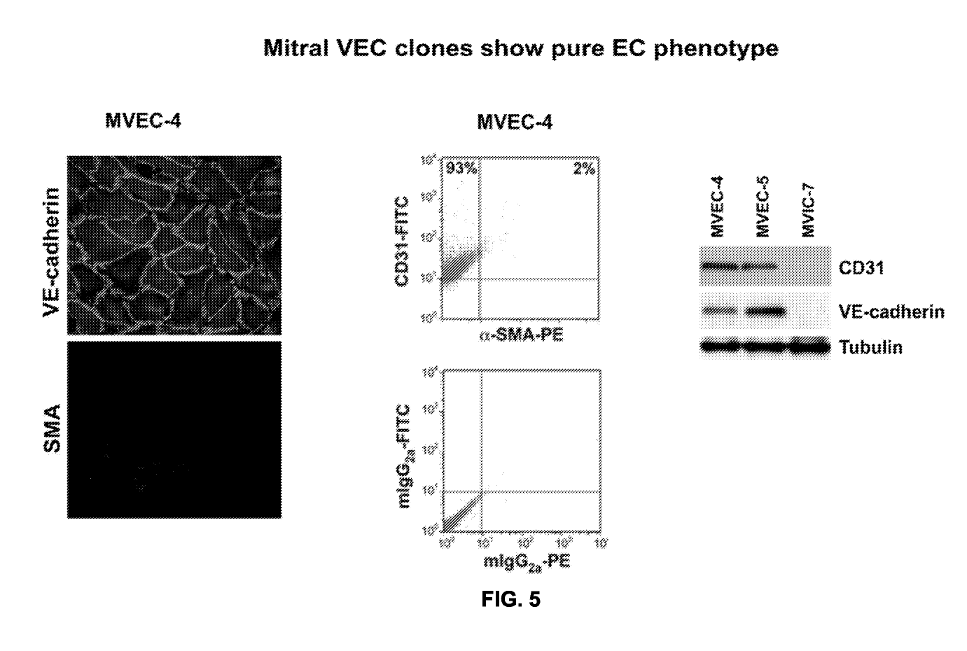

The co-expression of CD45 and αSMA in VE-cadherin-positive cells in IMI MVs suggested that these cells may be undergoing EndMT¹⁵ and that CD45 induction may coincide with EndMT processes. Our previous work showed increased EndMT, also called EMT, in tethered MVs in an ovine model, and suggested EndMT as a possible contributor to growth of MV leaflets to minimize MR⁵. To assess the link between EndMT and CD45, mitral VEC clones (prepared by expansion from a single mitral VEC11) and CAECs (incapable of undergoing EndMT11) were tested for CD45 expression in response to TGFβ1. In vitro treatment of mitral VEC clone E10 with TGFβ1 led to strong induction of CD45 and αSMA, detected by flow cytometry (FIGS. 3A and 3B). Interestingly, 23-25% of the mitral VEC clone E10 cells showed a low level of CD45 expression prior to TGFβ treatment. TGFβ-treated and non-treated CAECs did not express CD45 or αSMA (FIGS. 3C and 3D). Analysis of four additional mitral VEC clones, treated with TGFβ1, showed 19-88% of the cells were positive for CD45 (FIG. 3E). In non-treated TGFβ1 VEC clones, CD45-positive cells ranged from 3-40% (data not shown). In total, seven mitral VEC clones were studied: CD45 was significantly increased after 4 day exposure to TGFβ1 (P=0.029 by paired t-test). α-SMA was also significantly increased in TGFβ1-treated mitral VEC clones, as expected (P=0.007 by paired t-test) (data not shown). These results demonstrate that purified mitral VECs express CD45, and the levels are substantially increased by TGFβ. To determine if other hematopoietic markers were increased in TGFβ1-treated mitral VEC, we analyzed expression of CD11b (expressed on monocytes, neutrophils, natural killer cells, granulocytes and macrophages) and CD14 (expressed on macrophages, neutrophils and dendritic cells) by flow cytometry (data not shown). No expression was detected, while CD45 was increased as expected. These results demonstrate that purified mitral VECs specifically express CD45, and the levels are significantly increased by TGFβ1.

CD45 PTPase Inhibitor Blocks Expression of EndMT and Fibrosis Markers—

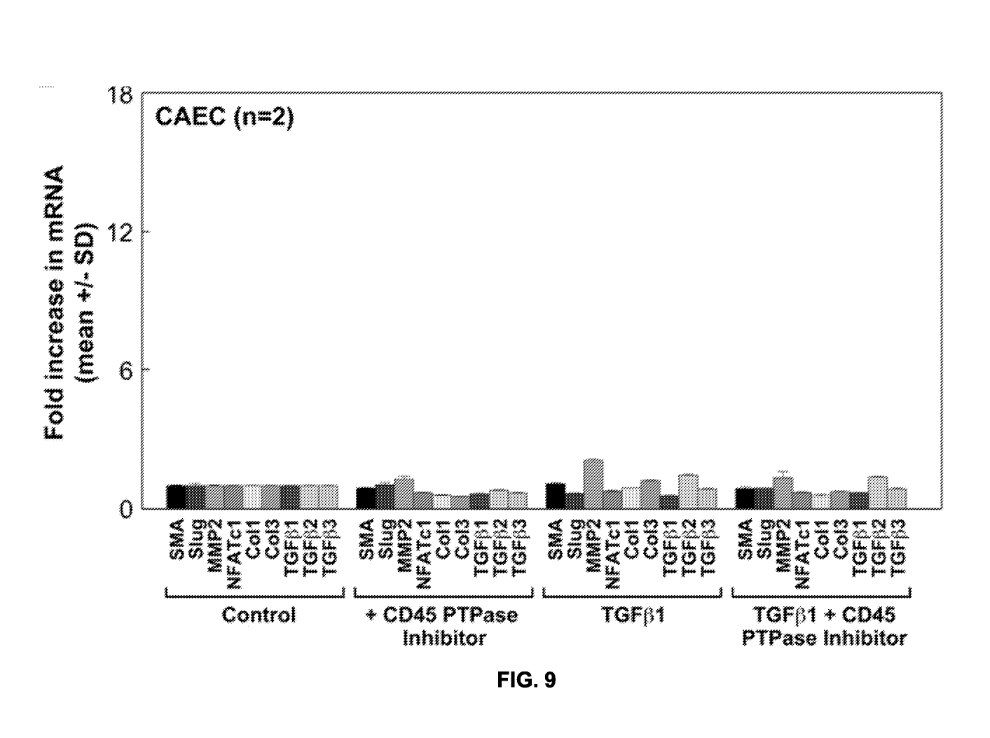

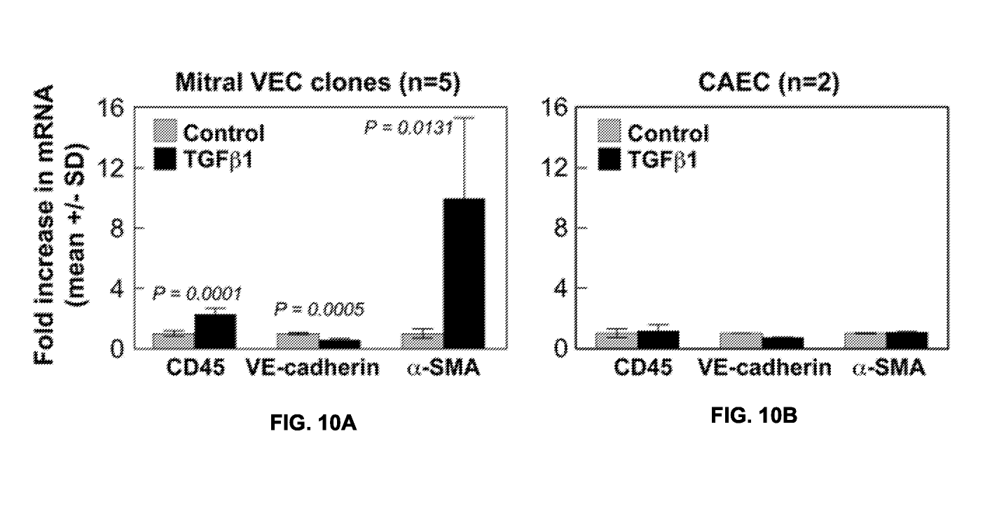

To verify the increased CD45 detected by flow cytometry, we analyzed CD45 mRNA in TGFβ1-treated mitral VEC and CAEC by qPCR (FIGS. 10A and 10B). In parallel, VE-cadherin and αSMA were measured to assess EndMT. Data compiled from five different mitral VEC clones showed significant increases in CD45 (p=0.0001) and αSMA mRNA (p=0.0131) (FIG. 10A). No changes in CD45, VE-cadherin or αSMA mRNA levels were seen in TGFβ1-treated CAEC (FIG. 10B). Well-established EndMT markers Slug and MMP2³¹′ ³², and NFATc1, which is negatively correlated with EndMT³³, were modulated as expected in TGFβ1-treated mitral VEC, consistent with our previous study”. Collagen 1 and collagen 3 mRNA transcripts as well as TGFβ1, TGFβ2 and TGFβ3 were also increased in TGFβ1-treated mitral VEC clones (FIG. 8). Inclusion of a CD45-selective protein tyrosine phosphatase (PTPase) (0.5 μM) during the 4 day TGFβ1 treatment significantly reduced αSMA, Slug, MMP-2, collagen 1, collagen 3, TGFβ1, TGFβ2 and TGFβ3 mRNA levels and restored NFATc1 mRNA (FIG. 8). P-values for FIG. 8 are shown Table 2. In contrast, no changes in these markers were seen in TGFβ1-treated CAEC, in the presence or absence of the CD45 PTPase inhibitor (FIG. 9). These results suggest that CD45 plays a functional role in EndMT and transition of mitral VEC to a fibrotic phenotype.

CD45 PTPase Inhibition Reduced EndMT-Associated Migration—

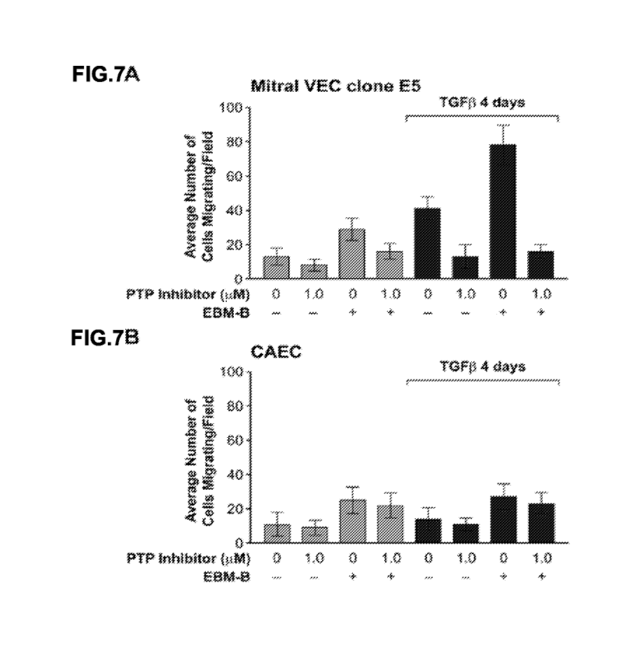

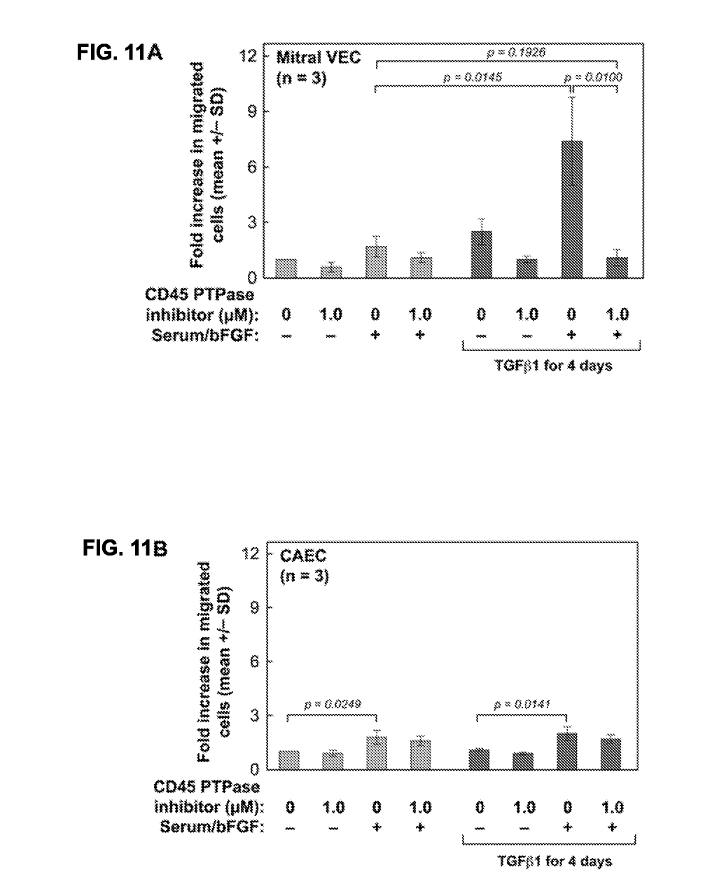

Increased migration is a hallmark of endothelial cells undergoing EndMT^{34,35}. Therefore, the inventors examined the requirement for ongoing CD45 phosphatase activity in migration of endothelial cells induced to undergo EndMT (FIGS. 7A, 7B, 11A and 11B). Mitral VEC and CAEC were treated without (gray bars) or with TGFβ1 for 4 days (black bars). Cells were then treated with or without CD45 PTPase inhibitor (1.0 μM) for 30 min, removed from culture dishes with trypsin, and resuspended in endothelial basal media (EBM) without serum and growth factors, +1.0 μM CD45 PTPase inhibitor. The cells were then assayed for migration towards EBM or EBM with serum and basic fibroblast growth factor (bFGF) added as chemoattractants²³. TGFβ-treated mitral VEC clones (n=3) showed significantly increased migration towards serum and bFGF (p=0.0145), which was significantly inhibited by the CD45 PTPase inhibitor (p=0.0100) (FIGS. 11A and 7A). Both control and TGFβ1-treated CAEC showed modest but significantly increased migration towards serum and bFGF in the 6 hour migration assay (p=0.0249; p=0.0141, respectively), but showed no increase in migration after TGFβ treatment and no response to the CD45 PTPase inhibitor (FIGS. 11B and 7B). An aliquot of mitral VEC and CAEC used for the migration assay was assayed for CD45 by flow cytometry to verify CD45 was expressed in the mitral VEC and not in the CAEC (data not shown). These results further indicate that CD45 plays a functional role in EndMT.

Increase in CD45+ Cells at 6 Months Post-IMI Correlates with MV Fibrosis and MR Severity—

To determine if CD45+MV cells associate with detrimental impacts, MVs were analyzed histologically and functionally. To assess collagen accumulation, which would stiffen the MV leaflets and impair their ability to form an effective seal to prevent MR, MV sections from sham (n=5) and IMI-6 month (n=5) sheep were analyzed by Masson Trichrome stain (FIG. 12A). Quantification of the percent positive area showed significantly increased collagen in the IMI-6 MVs (FIG. 12B), which correlated with increased CD45+ cells detected by immunohistochemistry (FIG. 12C). The excessive and disordered collagen detected by Masson Trichrome in the IMI-6 MV is a hallmark of fibrosis.

Total CD45+ cells in individual IMI-6 month MVs, measured by flow cytometry, showed a positive correlation with infarct surface area relative to the total left ventricular endocardial surface area (ESA) (R²=0.83, p=0.02) (FIG. 12D). The vena contracta width (VCW) indicating mitral regurgitation severity was measured in all sheep, sham (x) and IMI-6 month (▴), and plotted against CD45+ cells that increased 6 months after IMI. These CD45+ cells (VE-cadherin+/CD45+/αSMA+, VE-cadherin+/CD45+/α-SMA−, and VE-cadherin−/CD45+/αSMA+) showed a strong correlation (R²=0.72, p=0.002) with the VCW (FIG. 12E). Global LV remodeling was assessed by measuring the total LV ESA ratio between immediately post-MI (Ti) and 6 months (T2) by 3-dimensional echocardiography 16-19 This remodeling ratio showed a strong positive correlation with the MV CD45+ cells (R²=0.79, p<0.001). This suggests that not only the infarct size but left ventricular remodeling exerts an effect on the MV and in turn the number of CD45-expressing cells in the leaflets. The cell-modifying stimulus could be attributed to prolong cytokine release occurring after LV damage and failure, as has been described³⁶ CD45+ cells showed a negative correlation with ejection fraction (R²=0.71, p=0.002) (FIG. 12G).

In this study the inventors identify the novel capability of mitral VEC to express CD45 both in vivo post-MI and in vitro in response to TGFβ1. At 6 months post-MI, CD45+ endothelial cells were the most abundant CD45+ cell population in the MV, determined by flow cytometry of collagenase-digested anterior and posterior MV leaflets. Furthermore, the CD45+ endothelial cells co-expressed α-SMA, suggesting that these cells were undergoing EndMT. CD45+ hematopoietic cells and CD45+/αSMA+ fibrocytes were also detected and significantly increased in post-MI MVs. In vitro, ovine mitral VECs expressed a low level of endogenous CD45, which was increased significantly by TGFβ1 with a concomitant, significant increase in α-SMA.

CD45, a protein tyrosine phosphatase, is a transmembrane glycoprotein expressed on all differentiated hematopoietic cells, except erythrocytes and platelets. It is often used as a marker for cells of hematopoietic origin, and it is commonly used to isolate leukocytes. CD45 regulates a variety of cellular processes including cell proliferation, differentiation and migration¹⁶. Through its intrinsic phosphatase activity it can either dampen or activate signaling pathways by dephosphorylating Src kinase family members, in particular Lck in T cells and Lyn in B cells¹⁶. CD45 can also affect integrin-mediated adhesion in T cells and macrophages by down regulating the activity of Src family kinases Lyn and Hck, limiting their participation in the formation of stable focal adhesions¹⁶. CD45 has also been implicated in cell migration of hematopoietic cells^{16,18}.

CD45 is not normally expressed on endothelium or endothelial cells. The one exception is during embryonic development when specific sites within the yolk sac, the placenta and the dorsal aorta become “hemogenic” for a narrow window of time. CD45+/VE-cadherin+ cells bud from the hemogenic endothelium to give rise to hematopoietic stem cells^{19,20}. Purified cultures of human endothelial cells are typically devoid of CD45+ cells²¹, although we detected a limited window of hemogenic activity in human umbilical cord blood CD133-selected endothelial colony forming cells²². No CD45+ adult endothelium has been described so far. Therefore, our discovery of CD45+ endothelial cells in MVs 6 months post-IMI and in cultured mitral VECs is novel.

Endothelial CD45 is important in the MV adaptative response post-MI. The increased CD45, with its intrinsic phosphatase activity, may regulate adhesion and/or chemotaxis of the mitral VECs, as CD45 has been implicated in cellular adhesion and migration^{10,18,19}. Indeed, increased migration is a hallmark of cells undergoing EndMT¹⁵, which we speculate is an important process used to increase MV leaflet area and to replenish mitral VICs^{5,23}. The appearance of CD45+ mitral VECs undergoing EndMT (VE-cadherin+/CD45+/αSMA+ cells) at the expense of VE-cadherin+/CD45−/αSMA− cells represents the largest change observed in the MV at 6 months post MI (Table 1). Our in vitro results also suggest a potential link between EndMT and CD45, given the co-induction of CD45 and αSMA in TGFβ1-treated mitral VEC clones.

Approximately 15% of the CD45+ cells in the post-MI MVs may be fibrocytes, also known as myeloid fibroblasts¹³, based on presence of CD45 and αSMA and lack of VE-cadherin. CD45 indicates a hematopoietic origin of fibrocytes/myeloid fibroblasts and αSMA indicates myofibroblastic functionality¹⁴. Despite their relatively low number in IMI MVs, the infiltration of fibrocytes may be an important factor in the maladaptive MV adaptation response to MI, as these cells can release inflammatory and fibrogenic growth factors, including TNFα, IL6, IL8, TGFβ1-3, collagen I and III, and fibronectin²⁴ and importantly have been shown to contribute to cardiac fibrosis¹³. In myxomatous MVs, fibrocytes are increased under pro-fibrotic conditions²⁵, supporting their role in collagen deposition in the valves. Hajdu and colleagues identified a fibrocyte-like population in normal murine mitral valve leaflets that are spindle-shaped, CD45-positive and bone marrow-derived. They further characterized the cells as vimentin-positive but endothelial and leukocyte marker negative²⁶. They concluded that bone marrow-derived cells contribute to the VIC population under normal homeostatic conditions. The CD45+/αSMA+ that are increased the ovine IMI MVs may be related to this phenomenon, but would likely reflect an enhancement or exaggeration of the steady state influx of bone marrow cells into the MV. Whether some VE-cadherin+/CD45+/αSMA+ endothelial cells might downregulate VE-cadherin and become resident CD45+/αSMA+ fibrocytes is an open question that merits investigation.

In summary, here the inventors provide a relative quantification of multiple cell types in MVs at 6 months post-MI. (Table 1). The identification of a CD45+ endothelial population suggests upregulated endothelial CD45 may play a role in adaptation of MV cells post-MI. Given the plasticity of mitral VECs¹¹, CD45 may be an indicator of an adaptive phenotype. Finally, these results also forewarn against the use of CD45 as a marker for hematopoietic cells in the study of cardiac valves.

In order to determine if CD45 is required for EndMT in mitral VEC, we inhibited CD45 protein tyrosine phosphatase (PTP) activity in mitral VEC undergoing EndMT using two approaches. The first is a CD45-selective PTPase inhibitor²¹ (CalBiochem) and the second is siRNA-mediated knockdown of CD45. We have verified that the PTPase inhibitor does not affect mitral VEC viability up to 0.8 M (data not shown) and therefore is suitable for these experiments.

We used CD45 PTPase enzyme activity to verify each approach. PTPase activity in mitral VEC (+/−TGFβ) will be measured as follows: cell lysis using non-denaturing conditions, adsorption of CD45 using murine anti-ovine CD45 mAb (ABD Serotec) and ProteinG-Sepharose, PTPase assay using phosphatase substrates. Ovine lymphocytes and carotid artery endothelial cells will serve as positive and negative controls, respectively. We expect that mitral VEC treated with TGFβ for 4 days will have increased PTPase activity, which will then be titrated against the CD45 PTPase inhibitor. The expected IC50 is in the range of 0.2-3 M.

To test its effect on EndMT, the CD45 PTPase inhibitor will be added to mitral VEC treated+TGFβ for 4 days. EndMT will be evaluated by increased expression αSMA (western blot), as in previous publications¹⁰. We also examined the following EndMT markers: Slug, Snail1, MMP-2 and decreased NFATc1, measured by quantitative RT-PCR. Slug, Snail and MMP-2 are well-established markers of EndMT^{6,22}, while NFATc1 is negatively correlated with valve EndMT²³. We have used these validated markers in previous EndMT studies.

CD45 PTPase activity was measured in parallel in these experiments to verify effective inhibition over the 4 day assay. siRNA silencing of CD45 will be used as a complementary approach. Preliminary tests indicate mitral VEC tolerate and are transfected by the TransIT siRNA Transfection Reagent (Mirus). The siRNA approach has an advantage in that we may find inhibition of the phosphatase activity does not block EndMT but silencing CD45 expression does.

CD45 is Involved to Induce EndMT in CAEC—

Determine if CD45 is required for VEC migration and/or collagen deposition—Increased migration is a hallmark of EndMT as it enables the VEC to move into the interior of the leaflet as they differentiate into VIC. Some of the CD45-positive cells appear to be doing this. CD45 has been implicated neutrophil migration. We will use the same approaches described in to determine if CD45 is necessary and/or sufficient to increase the migratory capacity of mitral VEC using an endothelial migration assay and VEC migration assays known in the art.

Two types of migration assays were used. The first is a transwell assay in which the VEC will be plated in the upper chambers of a type I collagen-coated 6.5 mm Transwell™ inserts with 8.0 μm pore polycarbonate membranes. The lower chambers will contain media with or without serum, or with specific chemoattractants for VEC activated to undergo EndMT (PDGF-BB, bFGF)^{7,8}. See FIG. 6 for a schematic diagram of an embodiment of migration assay using a transwell. VEC will be allowed to migrate through 8.0 μM pores for 4 hours. Cells in the upper chamber that have not migrated will be removed and the cells that have migrated to the lower surface will be fixed with ice-cold methanol and stained with DAPI for visualization and quantification. In duplicate, cells will also be retrieved for biochemical analyses (e.g., qPCR). The second migration assay is a 3-dimensional collagen gel invasion in which VEC will be plated on top of a 0.4 ml gel of type I collagen in 24 well plates and allowed to invade for 72 hours. Migration of VEC through to the bottom of the gel will be evaluated using an inverted light microsope. Statistical analysis will be applied to quantified results.

The inventors will also analyze collagen deposition by CD45-positive cells, as a hallmark of fibrosis, and by CD45-inhibited cells. Specific collagens (I, II, III) will be measured by quantitative RT-PCR and overall collagen production and secretion can be measured by 3H-proline incorporation. We have experience with these assays on ovine VIC(Shapero et al, JMCC under revision). Briefly, for collagen biosynthesis, cells will be plated in 96 well plates in proline-free media, labeled with 1 uCi of 3H proline in proline-free DMEM, 1% GPS, 50 ug/ml L-ascorbic acid. Cell supernatants will be collected and protein precipitated with 10% trichloroacetic acid. Cell associated collagen will be quantified by washing the cell monolayers with PBS, removing cells from the plate with trypsin and precipitating with 10% trichloroacetic acid. The acid precipitated (protein incorporated) 3H counts will be detected using a Perkin Elmer Tri-Carb2900TR liquid scintillation analyzer. These experiments revealed that CD45 has a role for in post-EndMT migration and/or matrix deposition in the nascent VIC.

Additionally, the inventors showed that ecotopic activation of endogenous CD45 expression in human endothelial cells induces EndMT biomarkers, such as αSMA, in the cells. Human endothelial colony cells (ECFC) were transfected with CD45 lentiviral synergistic activation mediator (SAM) particles to transcriptionally activate the endogenous CD45 gene. Up to 42% of the transduced ECFC were expressing CD45, VE-cadherin, and αSMA, as determined by flow cytometry. (data not shown). This data show that in addition to mitral endothelial cells, expression of CD45 is sufficient to drive EndMT in human endothelial cells, and that inhibition of CD45 is a good strategy for inhibiting pathological EndMT.

The references cited herein and throughout the specification are incorporated herein by reference.

### REFERENCES

1. Levine R A, Schwammenthal E. Ischemic mitral regurgitation on the threshold of a solution: From paradoxes to unifying concepts. Circulation. 2005; 112:745-758
2. Walker G A, Masters K S, Shah D N, Anseth K S, Leinwand L A. Valvular myofibroblast activation by transforming growth factor-beta: Implications for pathological extracellular matrix remodeling in heart valve disease. Circ Res. 2004; 95:253-260
3. Grande-Allen K J, Barber J E, Klatka K M, Houghtaling P L, Vesely I, Moravec C S, McCarthy P M. Mitral valve stiffening in end-stage heart failure: Evidence of an organic contribution to functional mitral regurgitation. J Thorac Cardiovasc Surg. 2005; 130:783-790
4. Grande-Allen K J, Borowski A G, Troughton R W, Houghtaling P L, Dipaola N R, Moravec C S, Vesely I, Griffin B P. Apparently normal mitral valves in patients with heart failure demonstrate biochemical and structural derangements: An extracellular matrix and echocardiographic study. J Am Coll Cardiol. 2005; 45:54-61
5. Dal-Bianco J P, Aikawa E, Bischoff J, Guerrero J L, Handschumacher M D, Sullivan S, Johnson B, Titus J S, Iwamoto Y, Wylie-Sears J, Levine R A, Carpentier A. Active adaptation of the tethered mitral valve: Insights into a compensatory mechanism for functional mitral regurgitation. Circulation. 2009; 120:334-342
6. Kakio T, Matsumori A, Ono K, Ito H, Matsushima K, Sasayama S. Roles and relationship of macrophages and monocyte chemotactic and activating factor/monocyte chemoattractant protein-1 in the ischemic and reperfused rat heart. Lab Invest. 2000; 80:1127-1136
7. Brands M, Roelants J, de Krijger R, Bogers A, Reuser A, van der Ploeg A, Helbing W. Macrophage involvement in mitral valve pathology in mucopolysaccharidosis type vi (maroteaux-lamy syndrome). Am J Med Genet A. 2013; 161A:2550-2553
8. Molin D G, Bartram U, Van der Heiden K, Van Iperen L, Speer C P, Hierck B P, Poelmann R E, Gittenberger-de-Groot A C. Expression patterns of tgfbeta1-3 associate with myocardialisation of the outflow tract and the development of the epicardium and the fibrous heart skeleton. Dev Dyn. 2003; 227:431-444
9. Akhurst R J, Lehnert S A, Faissner A, Duffie E. Tgf beta in murine morphogenetic processes: The early embryo and cardiogenesis. Development. 1990; 108:645-656
10. Aikawa E, Nahrendorf M, Sosnovik D, Lok V M, Jaffer F A, Aikawa M, Weissleder R.

Multimodality molecular imaging identifies proteolytic and osteogenic activities in early aortic valve disease. Circulation. 2007; 115:377-386
11. Wylie-Sears J, Aikawa E, Levine R A, Yang J H, Bischoff J. Mitral valve endothelial cells with osteogenic differentiation potential. Arterioscler Thromb Vasc Biol. 2011; 31:598-607
12. Yamamoto K, de Waard V, Fearns C, Loskutoff D J. Tissue distribution and regulation of murine von willebrand factor gene expression in vivo. Blood. 1998; 92:2791-2801
13. Cieslik K A, Trial J, Crawford J R, Taffet G E, Entman M L. Adverse fibrosis in the aging heart depends on signaling between myeloid and mesenchymal cells; role of inflammatory fibroblasts. J Mol Cell Cardiol. 2014; 70:56-63
14. Duffield J S, Lupher M, Thannickal V J, Wynn T A. Host responses in tissue repair and fibrosis. Annu Rev Pathol. 2013; 8:241-276
15. Kovacic J C, Mercader N, Torres M, Boehm M, Fuster V. Epithelial-to-mesenchymal and endothelial-to-mesenchymal transition: From cardiovascular development to disease. Circulation. 2012; 125:1795-1808
16. Saunders A E, Johnson P. Modulation of immune cell signalling by the leukocyte common tyrosine phosphatase, cd45. Cell Signal. 2010; 22:339-348
17. Shivtiel S, Kollet O, Lapid K, Schajnovitz A, Goichberg P, Kalinkovich A, Shezen E, Tesio M, Netzer N, Petit I, Sharir A, Lapidot T. Cd45 regulates retention, motility, and numbers of hematopoietic progenitors, and affects osteoclast remodeling of metaphyseal trabecules. J Exp Med. 2008; 205:2381-2395
18. Lai J C, Wlodarska M, Liu D J, Abraham N, Johnson P. Cd45 regulates migration, proliferation, and progression of double negative 1 thymocytes. J Immunol. 2010; 185:2059-2070
19. Zape J P, Zovein A C. Hemogenic endothelium: Origins, regulation, and implications for vascular biology. Semin Cell Dev Biol. 2011; 22:1036-1047
20. Oberlin E, El Hafny B, Petit-Cocault L, Souyri M. Definitive human and mouse hematopoiesis originates from the embryonic endothelium: A new class of hscs based on ve-cadherin expression. Int J Dev Biol. 2010; 54:1165-1173
21. Yoder M C, Mead L E, Prater D, Krier T R, Mroueh K N, Li F, Krasich R, Temm C J, Prchal J T, Ingram D A. Redefining endothelial progenitor cells via clonal analysis and hematopoietic stem/progenitor cell principals. Blood. 2007; 109:1801-1809
22. Wu X, Lensch M W, Wylie-Sears J, Daley G Q, Bischoff J. Hemogenic endothelial progenitor cells isolated from human umbilical cord blood. Stem Cells. 2007; 25:2770-2776
23. Paranya G, Vineberg S, Dvorin E, Kaushal S, Roth S J, Rabkin E, Schoen F J, Bischoff J. Aortic valve endothelial cells undergo transforming growth factor-beta-mediated and non-transforming growth factor-beta-mediated transdifferentiation in vitro. American Journal of Pathology. 2001; 159:1335-1343
24. Pilling D, Fan T, Huang D, Kaul B, Gomer R H. Identification of markers that distinguish monocyte-derived fibrocytes from monocytes, macrophages, and fibroblasts. PLoS One. 2009; 4:e7475
25. Barth P J, Koster H, Moosdorf R. Cd34+ fibrocytes in normal mitral valves and myxomatous mitral valve degeneration. Pathol Res Pract. 2005; 201:301-304
26. Hajdu Z, Romeo S J, Fleming P A, Markwald R R, Visconti R P, Drake C J. Recruitment of bone marrow-derived valve interstitial cells is a normal homeostatic process. J Mol Cell Cardiol. 2011; 51:955-965.
27. Panchal R G, Ulrich R L, Bradfute S B, Lane D, Ruthel G, Kenny T A, Iversen P L, Anderson A O, Gussio R, Raschke W C, Bavari S. Reduced expression of cd45 protein-tyrosine phosphatase provides protection against anthrax pathogenesis. The Journal of biological chemistry. 2009; 284:12874-12885
28. Pfaffl M W. A new mathematical model for relative quantification in real-time rt-pcr. Nucleic Acids Res. 2001; 29:e45
29. Schmittgen T D, Livak K J. Analyzing real-time per data by the comparative c(t) method. Nat Protoc. 2008; 3:1101-1108
30. Willems E, Leyns L, Vandesompele J. Standardization of real-time per gene expression data from independent biological replicates. Anal Biochem. 2008; 379:127-129
31. Lincoln J, Yutzey K E. Molecular and developmental mechanisms of congenital heart valve disease. Birth Defects Res A Clin Mol Teratol. 2011; 91:526-534
32. Tao G, Kotick J D, Lincoln J. Heart valve development, maintenance, and disease: The role of endothelial cells. Curr Top Dev Biol. 2012; 100:203-232
33. Wu B, Wang Y, Lui W, Langworthy M, Tompkins K L, Hatzopoulos A K, Baldwin H S, Zhou B. Nfatc1 coordinates valve endocardial cell lineage development required for heart valve formation. Circulation research. 2011; 109:183-192
34. Person A D, Klewer S E, Runyan R B. Cell biology of cardiac cushion development. International Review of Cytology. 2005; 243:287-335
35. von Gise A, Pu W T. Endocardial and epicardial epithelial to mesenchymal transitions in heart development and disease. Circulation research. 2012; 110:1628-1645
36. Nian M, Lee P, Khaper N, Liu P. Inflammatory cytokines and postmyocardial infarction remodeling. Circulation research. 2004; 94:1543-1553
37. Handschumacher M D, Lethor J P, Siu S C, Mele D, Rivera J M, Picard M H, Weyman A E, Levine R A. A new integrated system for three-dimensional echocardiographic reconstruction: Development and validation for ventricular volume with application in human subjects. J Am Coll Cardiol. 1993; 21:743-753
38. Jiang L, Morrissey R, Handschumacher M D, Vazquez de Prada J A, He J, Picard M H, Weyman A E, Levine R A. Quantitative three-dimensional reconstruction of left ventricular volume with complete borders detected by acoustic quantification underestimates volume. Am Heart J. 1996; 131:553-559
39. Picard M H, Wilkins G T, Ray P A, Weyman A E. Natural history of left ventricular size and function after acute myocardial infarction. Assessment and prediction by echocardiographic endocardial surface mapping. Circulation. 1990; 82:484-494
40. Picard M H, Wilkins G T, Ray P A, Weyman A E. Progressive changes in ventricular structure and function during the year after acute myocardial infarction. Am Heart J. 1992; 124:24-31

TABLE 1
Table 1. Endothelial, interstitial and hematopoietic
cell populations in MVs from IMI infarcted sheep versus normal
sheep, percentage of total cells within each cell population.
Statistical significance measured using student t-test analysis.

| | | 6 months | |
|---|---|---|---|
| | Normal | IMI | Statistical |
| | n = 3 | n = 5 | Significance |
| VEC | 35.7 ± 9.5 | 4.6 ± 6.6 | P = 0.015 |
| VE-Cadherin⁺/CD45⁻/αSMA⁻ | | | |
| VEC^{CD45+} | 1.8 ± 2.3 | 9.0 ± 5.1 | P = 0.039 |
| VE-Cadherin⁺/CD45⁺/αSMA⁻ | | | |
| VEC-EndMT | 2.0 ± 1.4 | 3.8 ± 3.6 | P = 0.366 |
| VE-Cadherin⁺/CD45⁻/αSMA⁺ | | | |
| VEC^{CD45+}-EndMT | 0.4 ± 0.4 | 17.4 ± 4.6 | P = 0.001 |
| VE-Cadherin⁺/CD45+/αSMA⁺ | | | |
| Quiescent VIC | 59.8 ± 10.6 | 47.1 ± 12.8 | P = 0.187 |
| VE-Cadherin⁻/CD45⁻/αSMA⁻ | | | |
| Activated VIC | 0.1 ± 0.2 | 5.3 ± 5.1 | P = 0.084 |
| VE-Cadherin⁻/CD45⁻/αSMA⁺ | | | |
| Hematopoietic cells | 0.1 ± 0.1 | 8.8 ± 6.0 | P = 0.032 |
| VE-Cadherin⁻/CD45⁺/αSMA⁻ | | | |
| Fibrocytes | 0.0 ± 0.0 | 5.8 ± 2.9 | P = 0.011 |
| VE-Cadherin⁻/CD45⁺/αSMA⁺ | | | |

TABLE 2
Student t-tests performed on fold increases (mean ± SD) in FIG. 8.

| | | | | | | | | | |
|---|---|---|---|---|---|---|---|---|---|
| VE-cadherin | αSMA | Slug | MMP2 | NFATc1 | Collagen1 | Collagen3 | TGFβ1 | TGFβ2 | TGFβ3 |

Control vs TGFβ1 (p values)

| | | | | | | | | | |
|---|---|---|---|---|---|---|---|---|---|
| 0.0005 | 0.0131 | 0.0009 | 0.0133 | 0.0095 | 0.0009 | 0.0079 | 0.0019 | 0.00001 | 0.0082 |

TGFβ1 vs TGFβ1 + PTPase (p values)

| | | | | | | | | | |
|---|---|---|---|---|---|---|---|---|---|
| 0.1666 | 0.0143 | 0.0060 | 0.0196 | 0.0022 | 0.0115 | 0.0179 | 0.0344 | 0.00005 | 0.0051 |

TABLE 3

| Sheep ID | EDV (mL) | ESV (mL) | SV (mL) | EF (%) | VC (mm) |
|---|---|---|---|---|---|
| 6 months IMI | | | | | |
| 2082 | 94 | 51 | 43 | 45 | 4.6 |
| 2077 | 119 | 90 | 29 | 25 | 7.4 |
| 2057 | 97 | 53 | 43 | 45 | 4.1 |
| 2102 | 88 | 46 | 42 | 48 | 3.3 |
| 2071 | 81 | 42 | 38 | 48 | 4.1 |
| Sham | | | | | |
| 2095 | 54 | 16 | 38 | 70 | 1.4 |
| 2137 | 53 | 21 | 32 | 60 | 0 |
| 4048 | 49 | 15 | 34 | 69 | 0.0 |
| 4064 | 54 | 17 | 37 | 69 | 1.0 |
| 4033 | 47 | 14 | 33 | 69 | 1.1 |
| Normal | | | | | |
| 2115 | 69 | 28 | 41 | 59 | 0.17 |
| 3011 | 57 | 21 | 36 | 64 | 0.00 |
| 5149• | 105 | 51 | 54 | 51 | 0.00 |

•Sheep 5149 was larger (67 kg) than other sheep in the study (~45 kg), which could account for the larger LV

Echocardiographic measurements ischemic, sham and normal sheep. Volumes were calculated using three-dimensional data (full volume acquisition) on an Echomachine Philips IE33, using Philips Qlab software. EDV=End-diastolic volume; ESV=End-systolic volume; SV=Stroke volume; EF=Ejection fraction; VC=Vena contracta of the MR jet (proximal dimension reflecting orifice size). Sheep 2095 and 2137 were analyzed at 6 months and 4048, 4064, 4033 at 2 months.

What is claimed:

1.-9. (canceled)

10. A composition comprising an inhibitor of CD45 and an inhibitor of TGF-beta.

11. (canceled)

12. The composition of claim 10, wherein the composition further comprises an inhibitor of tissue fibrosis, wherein the inhibitor of tissue fibrosis (anti-fibrosis agent) is not a CD45 inhibitor or a TGFβ inhibitor.

13. A method of inhibiting pathological endothelial-to-mesenchymal transition (EndMT) comprising contacting an endothelial cell with an inhibitor of protein tyrosine phosphatase, receptor type, C, (PTPRC or CD45), or a composition comprising a CD45 inhibitor.

14. A method of inhibiting a pathological endothelial-to-mesenchymal transition (EndMT) in a mammal comprising administering an effective amount of an inhibitor of protein tyrosine phosphatase, receptor type, C, (PTPRC or CD45), or a composition comprising a CD45 inhibitor to the mammal.

15. The method of claim 13, wherein the pathological EndMT occurs in a disease or injury.

16. The method of claim 13, wherein the pathological EndMT produces excessive or undesirable tissue remodeling.

17. The method of claim 13, wherein the pathological EndMT produces excessive or undesirable fibrosis.

18. The method of claim 13, wherein the pathological EndMT occurs in conditions selected from the group consisting of mitral regurgitation (MR), myocardial infarction (MI), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, fibrosis induced transplant antibody mediated rejection, cerebral cavernous malformations, arteriosclerosis, intimal hyperplasia in vein-to-arterial graft and pulmonary fibrosis.

19. The method of claim 13, the method further comprising detecting the expression of CD45 in the endothelial cells.

20. The method of claim 13, the method further comprising detecting the presence of EndMT.

21. The method of claim 20, wherein the detection of EndMT comprises assessing expression of at least one biomarker selected from the group consisting of Fascin1, Vimentin and Hsp47.

22. The method of claim 13, wherein the CD45 inhibitor is targeted to an endothelial cell, wherein the endothelial cell is undergoing EndMT.

23. The method of claim 13, wherein the CD45 inhibitor is targeted to endothelial cells expressing CD45.

24. The method of claim 13, the method further comprising contacting the endothelial cell with a TGFβ inhibitor.

25. The method of claim 13, the method further comprising contacting the endothelial cell or administering to the mammal an anti-fibrosis agent wherein the anti-fibrosis agent is not a CD45 inhibitor or a TGFβ inhibitor.

26. The method of claim 14, wherein the pathological EndMT occurs in a disease or injury.

27. The method of claim 14, wherein the pathological EndMT produces excessive or undesirable tissue remodeling.

28. The method of claim 14, wherein the pathological EndMT produces excessive or undesirable fibrosis.

29. The method of claim 14, wherein the pathological EndMT occurs in conditions selected from the group consisting of mitral regurgitation (MR), myocardial infarction (MI), systemic sclerosis (SSc), fibrodysplasia ossificans progressive (FOP), cancer, fibrotic diseases, atrial fibrosis, cardiac fibrosis, diabetes mellitus-induced cardiac fibrosis, renal fibrosis, fibrosis induced transplant antibody mediated rejection, cerebral cavernous malformations, arteriosclerosis, intimal hyperplasia in vein-to-arterial graft and pulmonary fibrosis.

30. The method of claim 14, the method further comprising administering to the mammal with a TGFβ inhibitor.


SEQUENCE LISTING (2 sequences)

SEQ ID NO: 1 - DNA, 21 bp, Artificial Sequence
  Description of Artificial Sequence Synthetic oligonucleotide
  Description of Combined DNA/RNA Molecule Synthetic oligonucleotide
cuggcugaauuucagagcatt

SEQ ID NO: 2 - DNA, 21 bp, Artificial Sequence
  Description of Artificial Sequence Synthetic oligonucleotide
  Description of Combined DNA/RNA Molecule Synthetic oligonucleotide
ugcucugaaauucagccagtt